(19) United States
(12) Patent Application Publication
(10) Pub. No.: US 2020/0292542 A1
(43) Pub. Date: Sep. 17, 2020

(54) LATERAL FLOW ASSAY AND METHODS FOR DETECTING HIGH CONCENTRATION ANALYTES

(71) Applicant: Becton, Dickinson and Company, Franklin Lakes, NJ (US)

(72) Inventor: Guohong Liu, San Diego, CA (US)

(21) Appl. No.: 16/890,372

(22) Filed: Jun. 2, 2020

Related U.S. Application Data
(63) Continuation of application No. PCT/US2018/063586, filed on Dec. 3, 2018.
(60) Provisional application No. 62/594,974, filed on Dec. 5, 2017.

(51) Int. Cl. G01N 33/558; G01N 21/84

(52) U.S. Cl. CPC G01N 33/558; G01N 21/8483

(57) ABSTRACT

Sandwich-type lateral flow assay devices, kits, systems, and methods described herein include antibody-conjugated oversized particles that bind to analyte of interest in a sample and remain upstream of the capture zone when a fluid sample is applied to a lateral flow test device. Embodiments of the antibody-conjugated oversized particles allow for the precise determination of concentration of the analyte in the sample, including analyte present at high and very high concentrations. Lateral flow assays of the present disclosure can address drawbacks associated with the hook effect of sandwich-type lateral flow assays by eliminating the phase of the dose response curve where signal intensity is decreasing.

### CROSS REFERENCE TO RELATED APPLICATION

This application is a continuation of PCT Application No. PCT/US2018/063586, filed Dec. 3, 2018, which claims the benefit of U.S. Provisional Application No. 62/594,974, filed Dec. 5, 2017, each of which is hereby incorporated by reference in its entirety.

### FIELD

The present disclosure relates in general to lateral flow assay devices, kits, test systems, and methods. More particularly, the present disclosure relates to lateral flow assay devices to determine the concentration of analyte in a sample, including when the analyte of interest is present at high concentrations.

### BACKGROUND

Immunoassay systems, including lateral flow assays described herein provide reliable, inexpensive, portable, rapid, and simple diagnostic tests. Lateral flow assays can quickly and accurately detect the presence or absence of, and in some cases quantify, an analyte of interest in a sample. Advantageously, lateral flow assays can be minimally invasive and used as point-of-care testing systems. Lateral flow assays have been developed to detect a wide variety of medical or environmental analytes. In a sandwich format lateral flow assay, a labeled antibody against an analyte of interest is deposited on a test strip in or near a sample receiving zone. The labeled antibody may include, for example, a detector molecule or “label” bound to the antibody. When the sample is applied to the test strip, analyte present in the sample is bound by the labeled antibody, which flows along the test strip to a capture zone, where an immobilized antibody against the analyte binds the labeled antibody-analyte complex. The antibody immobilized on the capture line may be different than the labeled antibody deposited in or near the sample receiving zone. The captured complex is detected, and the presence of analyte is determined. In the absence of analyte, the labeled antibody flows along the test strip but passes by the capture zone. The lack of signal at the capture zone indicates the absence of analyte. The sandwich lateral flow assay, however, suffers from many disadvantages, including false negatives, inaccurate quantification, and lack of resolution when the analyte of interest is present in the sample at high concentrations.

### SUMMARY

It is therefore an aspect of this disclosure to provide improved lateral flow assays that precisely measure the concentration of an analyte of interest in a sample, including when the analyte is present in the sample at high concentrations.

Some embodiments disclosed herein relate to an assay test strip including a flow path configured to receive a fluid sample; a sample receiving zone coupled to the flow path; a capture zone; a labeled antibody or fragment thereof; and oversized particles in the flow path upstream of the capture zone. The capture zone is coupled to the flow path downstream of the sample receiving zone and including an immobilized capture agent specific to an analyte of interest. The labeled antibody or fragment thereof is coupled to the flow path upstream of the capture zone specific to the analyte of interest. The oversized particles are conjugated to an antibody or fragment thereof specific to the analyte of interest to form antibody-conjugated oversized particles of a size and dimension to remain upstream of the capture zone when the fluid sample is received on the assay test strip. In some embodiments, the flow path is configured to receive a fluid sample including the analyte of interest. In some embodiments, the labeled antibody or fragment thereof and the antibody-conjugated oversized particles compete to specifically bind the analyte of interest. In some embodiments, the labeled antibody or fragment thereof is configured to flow with bound analyte of interest in the flow path to the capture zone when the fluid sample is received on the assay test strip. In some embodiments, the labeled antibody bound to the analyte of interest is captured at the capture zone and emits a detectable signal.

In some embodiments, the flow path is configured to receive a fluid sample that does or does not include analyte of interest. In some embodiments, the antibody-conjugated oversized particles specifically bind to a known quantity of analyte of interest, thereby retaining a known quantity of analyte of interest upstream of the capture zone.

In further embodiments, the assay test strip includes a control zone downstream of the capture zone. In some embodiments, the control zone includes antibody that specifically binds to the labeled antibody or fragment thereof that does not bind to analyte of interest and flows past the capture zone. In some embodiments, when the fluid sample does not include an analyte of interest, the labeled antibody or fragment thereof flows to the control zone and emits an optical signal at the control zone only, indicating absence of the analyte of interest in the fluid sample. In some embodiments, the immobilized capture agent includes an antibody or a fragment thereof specific to the analyte of interest. In some embodiments, the antibody-conjugated oversized particles are integrated onto a surface of the test strip. In some embodiments, the oversized particles include gold particles, latex beads, magnetic beads, or silicon beads. In some embodiments, the oversized particle is about 1 μm to about 15 μm in diameter. In some embodiments, the fluid sample is selected from the group consisting of a blood, plasma, urine, sweat, or saliva sample. In some embodiments, the analyte of interest includes C-reactive protein (CRP) and the antibody or fragment thereof conjugated to the oversized particle includes an anti-CRP antibody or fragment thereof bound to the CRP.

Other embodiments disclosed herein relate to a kit including an assay test strip described above, including a flow path configured to receive a fluid sample; a sample receiving zone coupled to the flow path; a capture zone coupled to the flow path downstream of the sample receiving zone and including an immobilized capture agent specific to an analyte of interest; a labeled antibody or fragment thereof coupled to the flow path upstream of the capture zone specific to the analyte of interest; and oversized particles conjugated to an antibody or fragment thereof specific to the analyte of interest to form antibody-conjugated oversized particles that are about 250 times the size of the labeled antibody or fragment thereof.

Other embodiments disclosed herein relate to a diagnostic test system including an assay test strip or a kit described above; a reader including a light source and a detector; and a data analyzer.

Further embodiments disclosed herein relate to a method of determining a concentration of analyte of interest in a fluid sample. The method includes applying the fluid sample to an assay test strip described above; binding analyte present in the fluid sample to labeled antibody or fragment thereof; binding analyte present in the fluid sample to the antibody-conjugated oversized particles; flowing the fluid sample and labeled antibody bound to analyte in the flow path to the capture zone while the antibody-conjugated oversized particles bound to analyte does not flow in the flow path to the capture zone; binding the labeled antibody bound to analyte to the immobilized capture agent in the capture zone; detecting a signal from the labeled antibody bound to analyte immobilized in the capture zone; and determining the concentration of analyte based at least on the detected signal. In some embodiments, the concentration is determined based on the detected signal and the quantity of antibody-conjugated oversized particles on the assay test strip. In some embodiments, the detected signal is an optical signal, a fluorescence signal, or a magnetic signal. In some embodiments, the method further includes displaying an indication that the analyte of interest is present in the fluid sample. In some embodiments, the method further includes displaying a quantity of analyte of interest in the fluid sample. In some embodiments, the method further includes displaying an indication that the analyte of interest is present in elevated amounts.

Additional embodiments disclosed herein relate to a method of determining a concentration of analyte of interest in a fluid sample. In some embodiments, the method includes contacting the fluid sample with oversized particles that have been conjugated to an antibody or fragment thereof specific to the analyte of interest to form antibody-conjugated oversized particles; binding analyte of interest in the fluid sample to the antibody-conjugated oversized particles; after binding, applying the fluid sample having antibody-conjugated oversized particles to an assay test strip as described herein; and flowing the fluid sample and labeled antibody in the flow path to the capture zone. In some embodiments, if excess analyte of interest remains unbound to the antibody-conjugated oversized particles, the excess analyte of interest binds to labeled antibody or fragment thereof and flows through the flow path to the capture zone, where it is bound to the immobilized capture agent in the capture zone and emits a signal. In some embodiments, the assay test strip includes a flow path configured to receive a fluid sample, a sample receiving zone coupled to the flow path, a capture zone coupled to the flow path downstream of the sample receiving zone and including an immobilized capture agent specific to an analyte of interest, and a labeled antibody or fragment thereof coupled to the flow path upstream of the capture zone specific to the analyte of interest.

Some embodiments disclosed herein relate to a method of manufacturing an assay test strip. In some embodiments, the method includes coupling a sample receiving zone to a flow path configured to receive a fluid sample; coupling a capture zone to the flow path downstream of the sample receiving zone; coupling a labeled agent to the flow path upstream of the capture zone, and coupling oversized particles to the flow path. In some embodiments, the labeled agent includes a label and an antibody that specifically binds the analyte of interest. In some embodiments, the oversized particles conjugated to an antibody or fragment thereof specific to the analyte of interest to form antibody-conjugated oversized particles.

In some embodiments, coupling the labeled agent to the flow path includes forming a bond between the labeled agent and the flow path that breaks in the presence of fluid sample in the flow path. In some embodiments, coupling the oversized particles includes spraying a solution including the oversized particles onto a surface of the sample receiving zone. In some embodiments, coupling the oversized particles includes spraying a solution including the oversized particles onto a surface of the assay test strip between the sample receiving zone and the capture zone. In some embodiments, coupling the oversized particles includes applying a fluid solution including the oversized particles onto a surface of the assay test strip; and drying the fluid solution. In some embodiments, coupling includes integrating the oversized particles into a surface of the assay test strip.

In some embodiments, the method further includes immobilizing a capture agent specific to the analyte of interest on the capture zone. In some embodiments, the method further includes providing a solution including the oversized particles conjugated to an antibody or fragment thereof specific to the analyte of interest. In some embodiments, the analyte of interest includes C-reactive protein (CRP) and the labeled agent and the antibody-conjugated oversized particles include an antibody including anti-CRP antibody or a fragment of anti-CRP antibody. Still further embodiments disclosed herein relate to assay test strips made by the methods described above.

### BRIEF DESCRIPTION OF THE DRAWINGS

FIGS. 1A and 1B illustrate an example sandwich-type lateral flow assay before and after a fluid sample is applied at a sample receiving zone.

FIG. 2 illustrates an example dose response curve for the lateral flow assay of FIGS. 1A and 1B.

FIGS. 3A and 3B illustrate an example competitive-type lateral flow assay before and after a fluid sample is applied at a sample receiving zone.

FIG. 4 illustrates an example dose response curve for a competitive lateral flow assay of FIGS. 3A and 3B.

FIGS. 5A and 5B illustrate an example lateral flow assay according to a first embodiment of the present disclosure, before and after a fluid sample is applied at a sample receiving zone.

FIG. 6 illustrates an example lateral flow assay according to a second embodiment of the present disclosure, depicting the application of a sample to the lateral flow assay.

FIG. 7 illustrates an example dose response curve for the lateral flow assay of FIGS. 5A and 5B compared to a hook effect dose response curve.

### DETAILED DESCRIPTION

Devices, kits, systems, and methods described herein precisely determine the quantity of an analyte of interest in a sample, for example a concentration of the analyte in a sample of known volume. Advantageously, lateral flow devices, test systems, and methods according to the present disclosure precisely determine the quantity of an analyte of interest in situations where the analyte of interest is present in the sample at an elevated or “high” concentration. Lateral flow assays described herein can improve the resolution of a signal indicative of analyte concentration. Embodiments of the devices, kits, systems, and methods described herein may include a lateral flow assay and oversize particles conjugated to a binding agent specific to the analyte of interest. The binding agent can include, for example, an analyte-specific antibody or fragment thereof. Implementations of the oversized particles are referred to as “antibody-conjugated oversize particles” throughout the present disclosure as one example implementation, but it will be understood that oversized particles according to the present disclosure can be conjugated to any suitable binding agent that is specific to the analyte of interest, such as but not limited to an analyte-specific antibody or fragment thereof In one embodiment, antibody-conjugated oversize particles can be pre-mixed with a sample suspected of including analyte of interest and then added to a sample well of the lateral flow assay. In a second embodiment, antibody-conjugated oversize particles can be integrated in the lateral flow assay before the sample suspected of including analyte of interest is added to the lateral flow assay.

According the present disclosure, the antibody-conjugated oversize particles are relatively large in size compared to labeled agents (such as detector particles including colloidal gold, as one example) commonly used in lateral flow assays. In one non-limiting example, embodiments of the antibody-conjugated oversize particles described herein can be around 10 μm in diameter, while a commonly-used labeled agent is typically around 40 nm in diameter. In contrast to the labeled agent that can easily move from the sample well and through the membrane of the lateral flow assay when a fluid sample is added, embodiments of the antibody-conjugated oversize particles described herein do not mobilize through the membrane when a fluid sample is added to the lateral flow assay. Instead, embodiments of the antibody-conjugated oversize particles described herein capture analyte in the sample (if present) in the sample well upon sample application and retain the captured analyte in the sample well. As a result, the embodiments of the antibody-conjugated oversize particles described herein reduce the amount of analyte that can form label-antibody-analyte complex and flow through the membrane to a capture region of the lateral flow assay to generate a detectable signal. The decreased amount of label-antibody-analyte complex reaching the capture region of the lateral flow assay results in a dose response curve exhibiting a single, rising phase.

Embodiments described herein thus solve drawbacks associated with the hook effect of conventional sandwich-type lateral flow assays by reducing or altogether eliminating the phase of the dose response curve where signals are decreasing. Advantageously, the single, rising phase exhibited by lateral flow assays of the present disclosure can be used to determine, with very high accuracy and reliability, a quantity of analyte in the sample, and in particular, the quantity of analyte when it occurs in high or very high concentrations. In some implementations described below, very accurate quantitative measurements are achieved because antibody-conjugated oversized particles according to the present disclosure can reliably retain a predictable, known quantity of bound analyte of interest upstream of the capture zone. Additionally, devices, systems, and methods of the present disclosure include lateral flow assays that can develop a detectable signal and generate a test result in as little as 2 minutes, in contrast to conventional lateral flow assays that require as many as 10 to 15 minutes to develop a detectable signal and generate a test result.

Embodiments of the present disclosure are described with reference to a lateral flow device. In some embodiments, the lateral flow device is implemented on a test strip, but other forms may be suitable. In the test strip format, a test sample fluid, suspected of containing an analyte, flows (for example by capillary action) through the strip. The strip may be made of any suitable material, including but not limited to bibulous materials such as paper, nitrocellulose, and cellulose. The sample fluid is received at a sample reservoir. The sample fluid may flow along the strip to a capture zone in which the analyte (if present) interacts with a capture agent to indicate a presence, absence, and/or quantity of the analyte. The capture agent can include antibody immobilized in the capture zone.

Embodiments of the present disclosure are described with reference to an optical signal generated at the capture zone of a lateral flow device, but other forms of detectable signals may be implemented in accordance with the present disclosure. Signals generated by assays according to the present disclosure may include an optical signal generated by reflectance-type labels (such as but not limited to gold nanoparticle labels), a fluorescent signal generated by fluorescence-type latex bead labels, magnetic field signals generated by magnetic nanoparticle labels that generate signals indicating a change in magnetic fields associated with the assay, or any other suitable signal.

According to the present disclosure, a lateral flow assay includes a labeled agent that is specific to an analyte of interest suspected of being present in a sample. The labeled agent is initially integrated onto a surface, for example onto a conjugate pad, of a lateral flow assay test strip at a sample well (or sample reservoir region). In some embodiments, the labeled agent is a labeled antibody or fragment thereof. In some embodiments, the labeled agent is labeled with a label that emits a detectable signal, for example, a metal nanoparticle, such as a gold nanoparticle, a colored latex, a fluorescent particle, or other label that emits a detectable signal.

Upon application of a fluid sample to the test strip at the sample reservoir, the labeled agent solubilizes into the fluid sample, and competes with the antibody-conjugated oversized particles for binding of analyte of interest in the sample. Analyte bound by the antibody-conjugated oversized particles is retained in a known amount (based on the quantity and binding capacity of the antibody-conjugated oversized particles applied to the test strip) and does not flow through the test strip. In contrast, analyte bound by labeled agent forms a label-agent-analyte complex, and travels to the capture zone of the test strip with the fluid sample. In some embodiments, the capture agent is an antibody or fragment thereof that is specific to the analyte of interest. The label-agent-analyte complex binds to capture agent in the capture zone, generating a signal indicative of a quantity of analyte in excess of the binding capacity of the antibody-conjugated oversized particles. The result is a single-phase dose response curve, where the signal intensity increases with an increase in concentration of analyte in excess of the antibody-conjugated oversized particles.

The antibody-conjugated oversized particles according to the present disclosure can be applied to the test strip in many ways. In one non-limiting example described in detail below, the antibody-conjugated oversized particles are initially integrated onto the surface of the test strip at the sample reservoir or label zone, before a sample is applied to the test strip. In another non-limiting example, sample is contacted with a quantity of antibody-conjugated oversized particles and then the sample mixed with antibody-conjugated oversized particles is applied to the test strip. The antibody-conjugated oversized particles is pre-integrated onto the test strip (or contacted with the sample) in a known quantity, such that a known quantity of analyte of interest is bound by the antibody-conjugated oversized particles. When the sample is applied to the test strip, the fluid front carries labeled agent bound to analyte of interest to the capture zone, but analyte bound to the antibody-conjugated oversized particles does not mobilize through the test strip and remains in place upstream up the capture zone (either in the zone where it was initially integrated on the test strip or in the sample well where it was deposited when the sample was applied to the test strip). In some cases, an optical signal detected at the capture zone is compared to a dose response curve specific to the test device and known quantity of antibody-conjugated oversized particles to determine a quantity of analyte of interest. In other cases, the quantity of analyte of interest is calculated directly by adjusting a dose response curve of a conventional lateral flow sandwich-type assay by the amount of analyte retained by antibody-conjugated oversized particles (which can be determined using the amount and conjugation ratio of the antibody-conjugated oversized particles used during the test).

Without being bound to any particular theory, the binding of analyte by the antibody-conjugated oversized particles, whether pre-integrated on the lateral flow device or contacted with the sample prior to application to the lateral flow device, decreases the amount of analyte that may be captured by labeling agent and detected at the capture zone, thereby increasing the resolution of the rising phase of the dose response curve, and generating a single-phase dose response curve by removing the second phase from the conventional sandwich-type lateral flow assay dose response curve. Lateral flow assays of the present disclosure solve drawbacks associated with the hook effect of sandwich-type lateral flow assays by eliminating the phase of the dose response curve where signals are decreasing. Additionally, compared to the portion of a conventional sandwich-type dose response curve where signals are increasing, resolution of signals in the ascending portion of dose response curves of the present disclosure is dramatically improved.

Signals generated by lateral flow assays described herein when the analyte is at high concentrations include many advantageous features. In example embodiments, signals that are generated when the analyte is at high concentration are readily detectable (for example, they have an intensity within a range of signals which conventional readers can typically discern and are well spaced apart), they do not overlap on the dose response curve with signals generated at zero or low concentrations, and they can be used to calculate a highly-accurate concentration reading at high and even very high concentrations. Embodiments of the lateral flow assays described herein avoid uncertainty associated with correlating a particular detected signal with a quantity of analyte (especially analyte at high concentration), such as uncertainty that occurs in reading sandwich-type lateral flow assays that generate a single optical signal corresponding to both a low concentration and a high concentration of analyte due to the hook effect. In contrast, lateral flow assays according to the present disclosure generate an optical signal that clearly and unambiguously corresponds to a zero or low concentration of analyte or a high concentration of analyte. In some cases, zero or low concentrations can be directly correlated to a normal or “healthy” level of analyte in the subject, and high concentrations of analyte can be directly correlated to a non-normal or “unhealthy” level of analyte in the subject.

Embodiments of the lateral flow assay described herein are particularly advantageous in diagnostic tests for analytes of interest that naturally occur at low concentrations in healthy individuals but elevate to high concentrations in individuals with a disease condition or disorder. Detection of relatively little or no signal correlates to zero to low concentration range where the operator only seeks to confirm that the analyte is present at a low concentration (indicator of healthy levels) and does not require specificity or resolution of signals, as the signal remains at or near zero until the binding capacity of the antibody-conjugated oversized particles is saturated. Once the antibody-conjugated oversized particles become saturated, readily-detectable, high resolution signals are generated in a single rising phase dose response curve, where the operator seeks to confirm that the analyte is present at high concentration (indicator of abnormal or disease condition) and in particular seeks to quantify the analyte of interest whenever it is present at high concentrations. The ability to accurately pinpoint the precise concentration of an analyte of interest when it is within a range of high concentrations can also allow the operator to ascertain the stage or progress of a disease or other condition in the subject, such as a mild stage or a severe stage.

Kits including a lateral flow assay and antibody-conjugated oversize particles; systems including a lateral flow assay, antibody-conjugated oversize particles, and a reader; and methods of using the lateral flow assay for determining a quantity of analyte in a sample are described herein. Various aspects of the lateral flow assays provide advantages over existing lateral flow assays. For example, in some embodiments, the lateral flow assays described herein can accurately determine the concentration of elevated analyte in a sample without the requirement to first dilute the sample. In addition, in some embodiments, the amount of antibody-conjugated oversized particles either placed on the lateral flow assay or placed in the sample prior to application on the lateral flow assay can be varied to accommodate the requirement of different concentration ranges of analytes.

Various aspects of the devices, test systems, and methods are described more fully hereinafter with reference to the accompanying drawings. The disclosure may, however, be embodied in many different forms. Based on the teachings herein one skilled in the art should appreciate that the scope of the disclosure is intended to cover any aspect of the devices, test systems, and methods disclosed herein, whether implemented independently of or combined with any other aspect of the present disclosure. For example, a device may be implemented or a method may be practiced using any number of the aspects set forth herein.

Although particular aspects are described herein, many variations and permutations of these aspects fall within the scope of the disclosure. Although some benefits and advantages are mentioned, the scope of the disclosure is not intended to be limited to particular benefits, uses, or objectives. Rather, aspects of the disclosure are intended to be broadly applicable to different detection technologies and device configurations some of which are illustrated by way of example in the figures and in the following description. The detailed description and drawings are merely illustrative of the disclosure rather than limiting, the scope of the disclosure being defined by the appended claims and equivalents thereof.

### Sandwich-Type and Competitive-Type Lateral Flow Assays

Lateral flow assays can be performed in a sandwich or competitive format. Sandwich and competitive format assays described herein will be described in the context of reflective-type labels (such as gold nanoparticle labels) generating an optical signal, but it will be understood that assays may include latex bead labels configured to generate fluorescence signals, magnetic nanoparticle labels configured to generate magnetic signals, or any other label configured to generate a detectable signal. Sandwich-type lateral flow assays include a labeled antibody deposited at a sample reservoir on a solid substrate. After sample is applied to the sample reservoir, the labeled antibody solubilizes in the sample, whereupon the antibody recognizes and binds a first epitope on the analyte in the sample, forming a label-antibody-analyte complex. This complex flows along the liquid front from the sample reservoir through the solid substrate to a capture zone (also referred to herein as a “test line”), where immobilized antibodies (sometimes referred to as “capture agent”) are located. In some embodiments, the immobilized antibodies may be the same antibody type as the labeled antibody. For example, in some cases where the analyte is a multimer or contains multiple identical epitopes on the same monomer, the labeled antibody deposited at the sample reservoir can be the same antibody type as the antibody immobilized in the capture zone. In some embodiments, the immobilized antibody may be a different antibody type, and recognizes a different epitope or recognition site on the analyte. The immobilized antibody recognizes and binds an epitope on the analyte, thereby capturing label-antibody-analyte complex at the capture zone.

The presence of labeled antibody at the capture zone provides a detectable optical signal at the capture zone. In one non-limiting example, metallic nanoparticles (gold, silver, copper, platinum, cadmium, palladium, or composites thereof) are used to label the antibodies because they are relatively inexpensive, stable, and provide easily observable color indications based on the surface plasmon resonance properties of metallic nanoparticles. In some cases, this signal provides qualitative information, such as whether or not the analyte is present in the sample. In some cases, this signal provides quantitative information, such as a measurement of the quantity of analyte in the sample.

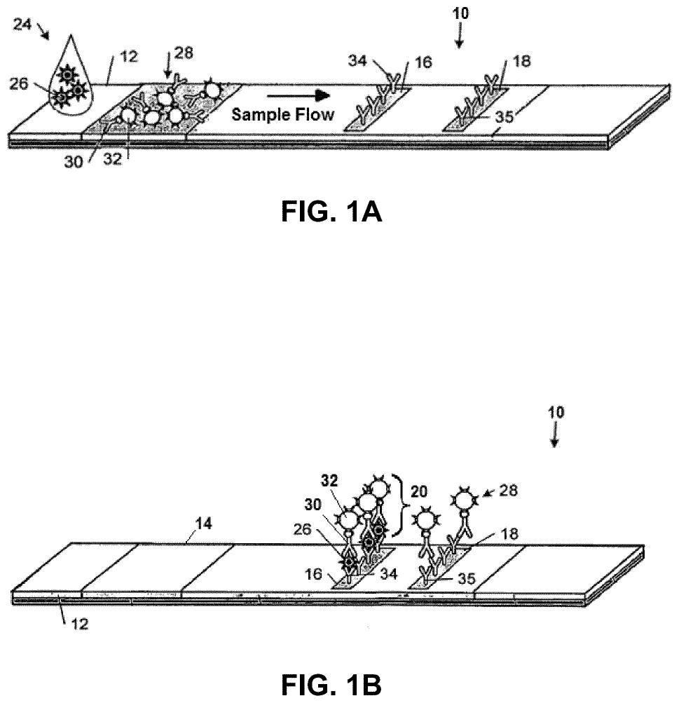

FIGS. 1A and 1B illustrate an example sandwich-type lateral flow device 10. The lateral flow device 10 includes a sample reservoir 12, a label zone 14, a capture zone 16, and a control line 18. FIGS. 1A and 1B illustrate the lateral flow device 10 before and after a fluid sample 24 has been applied to the sample reservoir 12. In the example illustrated in FIGS. 1A and 1B, the sample 24 includes analyte of interest 26. The label zone 14 that is in or near the sample reservoir 12 includes a labeled agent 28. In this example sandwich-type lateral flow device, the labeled agent 28 includes an antibody or antibody fragment 30 bound to a label 32. A capture agent 34 is immobilized in the capture zone 16. A control agent 35 is immobilized on the control line 18.

When the fluid sample 24 is applied to the sample reservoir 12, the sample 24 solubilizes the labeled agent 28, and the labeled agent 28 binds to analyte 26, forming an label-antibody-analyte complex 20. Accordingly, in the example sandwich-type lateral flow device 10, the label-antibody-analyte complex 20 is not formed until after the fluid sample 24 containing the analyte of interest 26 is applied to the lateral flow device. Further, in the example sandwich-type lateral flow device 10, the analyte in the label-antibody-analyte complex 20 is analyte from the fluid sample 24. As shown in FIG. 1B, this complex 20 flows through the test strip to the capture zone 16, where it is bound by the capture agent 34. The now-bound complex 20 (and specifically, the label 32 on the now-bound complex 20) emits a detectable optical signal at the capture zone 16.

Labeled agent 28 that did not bind to any analyte 26 passes through the capture zone 16 (there being no analyte 26 to bind to a capture agent 34 in the capture zone 16) and continues to flow down the lateral flow device 10. In lateral flow assays that include the control line 18 such as that illustrated here, the immobilized control agent 35 captures labeled agent 28 that did not bind to analyte 26 and passed through the capture zone 16 to the control line 18. In some embodiments, the control agent 35 captures the labeled agent 28 at the Fc region of the antibody. In some embodiments, the control agent 35 captures the labeled agent 28 at the Fab region of the antibody. This labeled agent 28 bound at the control line 18 emits a detectable optical signal that can be measured and used to indicate that the assay operated as intended (for example, the sample 24 flowed from the sample reservoir 12 and through the capture zone 16 as intended during normal operation of the lateral flow assay).

Lateral flow assays can provide qualitative information, such as information on the absence or presence of the analyte of interest in the sample. For example, detection of any measurable optical signal at the capture zone 16 can indicate that the analyte of interest is present in the sample (in some unknown quantity). The absence of any measurable optical signal at the capture zone can indicate that the analyte of interest is not present in the sample or below the detection limit. For example, if the sample 24 did not contain any analyte of interest 26 (not illustrated), the sample 24 would still solubilize the labeled agent 28 and the labeled agent 28 would still flow to the capture zone 16. The labeled agent 28 would not bind to the capture agent 34 at the capture zone 16, however. It would instead flow through the capture zone 16, through the control line 18, and, in some cases, to an optional absorbing zone. Some labeled agent 28 would bind to the control agent 35 deposited on the control line 18 and emit a detectable optical signal. In these circumstances, the absence of a measureable optical signal emanating from the capture zone 16 is an indication that the analyte of interest is not present in the sample 24, and the presence of a measureable optical signal emanating from the control line 18 is an indication that the sample 24 traveled from the sample receiving zone 12, through the capture zone 16, and to the capture line 18 as intended during normal operation of the lateral flow assay.

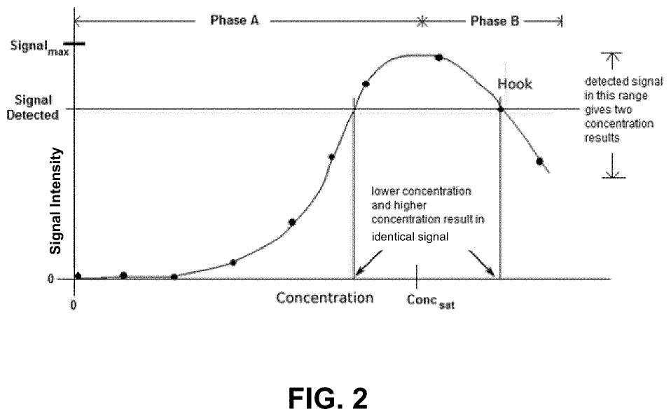

Some lateral flow devices can provide quantitative information, such as a measurement of the quantity of analyte of interest in the sample. The quantitative measurement obtained from the lateral flow device may be a concentration of the analyte that is present in a given volume of sample. FIG. 2 illustrates an example quantitative measurement obtained from the sandwich-type lateral flow assay illustrated in FIGS. 1A and 1B. FIG. 2 is a dose response curve that graphically illustrates the relationship between an intensity of a signal detected at the capture zone (measured along the y-axis) and the concentration of analyte in the sample (measured along the x-axis). Example signals include optical signals, fluorescence signals, and magnetic signals.

As shown by the first data point at zero concentration in FIG. 2, if the sample does not contain any analyte of interest, the concentration of analyte in the sample is zero and no analyte binds to the labeled agent to form a label-antibody-analyte complex. In this situation, there are no complexes that flow to the capture zone and bind to the capture antibody. Thus, no detectable optical signal is observed at the capture zone and the signal magnitude is zero.

A signal is detected as the concentration of analyte in the sample increases from zero concentration. As demonstrated by data points in Phase A, the signal increases with increased analyte concentration in the sample. This takes place because as the analyte concentration increases, the formation of label-antibody-analyte complex increases. Capture agent immobilized at the capture zone binds the increasing number of complexes flowing to the capture zone, resulting in an increase in the signal detected at the capture zone. In Phase A, the signal continues to increase as the concentration of the analyte in the sample increases.

In some instances, if a sample has a concentration of analyte that exceeds the amount of labeled agent available to bind to the analyte, excess analyte is present. In these circumstances, excess analyte that is not bound by labeled agent competes with the label-antibody-analyte complex to bind to the capture agent in the capture zone. The capture agent in the capture zone will bind to un-labeled analyte (in other words, analyte not bound to a labeled agent) and to label-antibody-analyte complex. Un-labeled analyte that binds to the capture agent does not emit a detectable signal, however. As the concentration of analyte in the sample increases in Phase B, the amount of un-labeled analyte that binds to the capture agent (in lieu of a label-antibody-analyte complex that emits a detectable signal) increases. As increasing amounts of un-labeled analyte bind to the capture agent in lieu of label-antibody-analyte complex, the signal detected at the capture zone decreases, as shown by data points in Phase B.

This phenomenon where the detected signal increases during Phase A and the detected signal decreases in Phase B is referred to as a “hook effect.” As the concentration of analyte increases in the Phase A, more analyte binds to the labeled agent, resulting in increased signal strength. At a point “Concₛₐₜ,” the labeled agent is saturated with analyte from the sample (for example, the available quantity of labeled agent has all or nearly all bound to analyte from the sample), and the detected signal has reached a maximum value Signalₘₐₓ. As the concentration of the analyte in the sample continues to increase in Phase B, there is a decrease in the detected signal as excess analyte above the labeled agent saturation point competes with the labeled agent-analyte to bind to the capture agent.

The hook effect, also referred to as “the prozone effect,” adversely affects lateral flow assays, particularly in situations where the analyte of interest is present in the sample at a concentration in Phase B. The hook effect can lead to inaccurate test results. For example, the hook effect can result in false negatives or inaccurately low results. Specifically, inaccurate results occur when a sample contains elevated levels of analyte that exceed the concentration of labeled agent deposited on the test strip. In this scenario, when the sample is placed on the test strip, the labeled agent becomes saturated, and not all of the analyte becomes labeled. The unlabeled analyte flows through the assay and binds at the capture zone, out-competing the labeled complex, and thereby reducing the detectable signal. Thus, the device (or the operator of the device) is unable to distinguish whether the optical signal corresponds to a low or a high concentration, as the detected signal corresponds to both a low and a high concentration. If analyte levels are sufficiently elevated, then the analyte completely out-competes the labeled complex, and no signal is observed at the capture zone, resulting in a false negative test result.

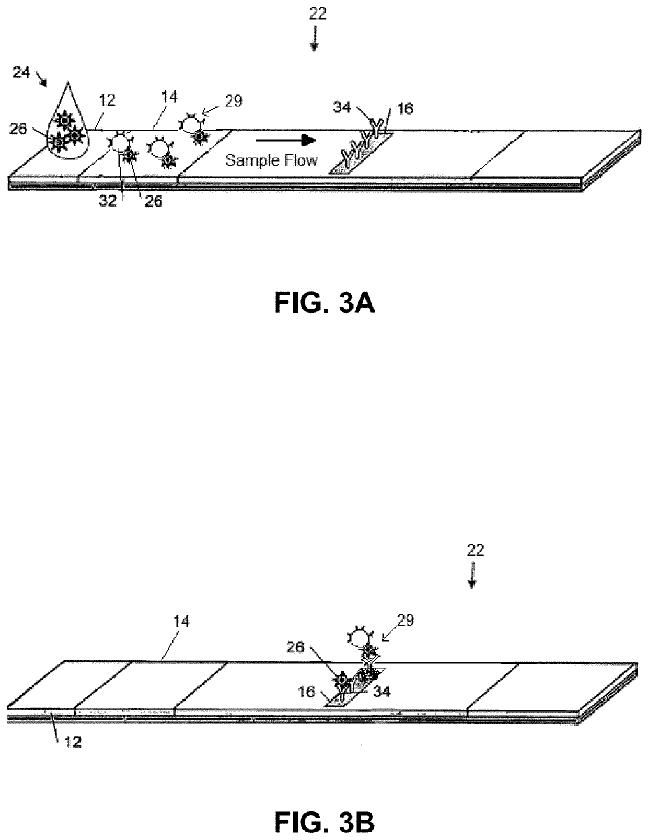

Inaccurate test results can also result from competitive-type lateral flow assays. In contrast to sandwich-type lateral flow assays, in a competitive-type lateral flow assay the un-labeled analyte of interest from a sample competes with labeled analyte of interest to bind to a capture agent at the capture zone. FIGS. 3A and 3B illustrate an example competitive-type lateral flow assay 22. The lateral flow device 22 includes a sample reservoir 12, a label zone 14, and a capture zone 16. FIGS. 3A and 3B illustrate the lateral flow device 22 before and after a fluid sample 24 has been applied to the sample reservoir 12. In the example illustrated in FIGS. 3A and 3B, the fluid sample 24 includes analyte of interest 26. The label zone 14 that is in or near the sample reservoir 12 includes a labeled agent 29. In this example competitive-type lateral flow device, the labeled agent 29 includes an analyte of interest 26 bound to a label 32. A capture agent 34 is immobilized in the capture zone 16.

The sample 24 that includes un-labeled analyte 26 is applied to the sample reservoir 12. The sample 24 solubilizes the labeled agent 29. The un-labeled analyte 26 in the sample 24 and the labeled agent 29 flow together to the capture zone 16, where both un-labeled analyte 26 from the sample 24 and labeled agent 29 bind to the capture agent 34 immobilized in the capture zone 16. As shown in FIG. 3B, the labeled agent and the un-labeled analyte 26 compete with each other to bind to a fixed amount of capture agent 34. Labeled agent 29 bound to capture agent 34 (and specifically, the label 32 in labeled agent 29) emits a detectable optical signal, whereas un-labeled analyte 26 that originated from sample 24 and bound to capture agent 34 does not emit a detectable optical signal.

Detection of an optical signal from the capture zone 16 can provide qualitative or quantitative information about the analyte of interest 26. In the case where fluid sample 24 does not include any analyte 26 (not illustrated), the sample 24 would still solubilize the labeled agent 29 and the labeled agent 29 would still flow to the capture zone 16. The capture agent 34 in the capture zone 16 will bind to labeled agent 29 (which does not compete with any un-labeled analyte from the sample), resulting in a detected optical signal of maximum intensity or near maximum intensity. In a case where the sample 24 includes analyte 26 at very low or low concentration, an optical signal of maximum intensity or near maximum intensity may also be detected. This is because the proportion of un-labeled analyte 26 bound to capture agent 34 to labeled agent 29 bound to capture agent 34 will be low. Thus, it may be difficult to determine if a detected optical signal at maximum intensity should correlate to zero concentration or low concentration of analyte 26 in the sample 24.

As the concentration of un-labeled analyte 26 increases in the sample 24, the detected optical signal emitted from the capture zone 16 decreases. This is because competition for the capture agent 34 increases with increasing analyte concentration in the sample, and the proportion of un-labeled analyte 26 bound to capture agent 34 to labeled agent 29 bound to capture agent 34 will progressively increase. If the analyte is present in the sample in high or very high concentrations, however, the optical signal detected at the capture zone 16 rapidly decreases to low magnitude signals. This rapid decrease in the strength of the optical signal as the concentration of analyte in the sample increases to high and very high concentrations makes it difficult if not impossible to precisely determine the concentration of the analyte, and in some cases renders the device inoperable to determine the concentration of the analyte at all. Competitive-type lateral flow devices such as that illustrated in FIGS. 3A and 3B are virtually incapable of accurately determining the precise concentration of the analyte of interest when the analyte of interest is present at high concentrations (for example, when the proportion of un-labeled analyte to labeled agent is high).

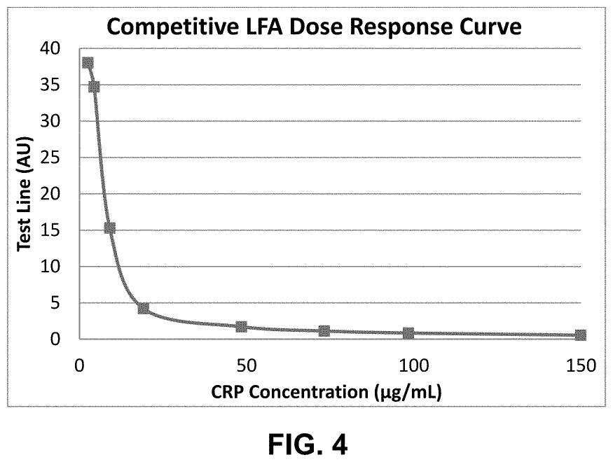

FIG. 4 illustrates a dose response curve generated in an example competitive-type lateral flow device such as that described above with reference to FIGS. 3A and 3B. As shown in FIG. 4, the dose response curve of a competitive-type lateral flow assay exhibits a steep decrease in signal in concentrations of analyte ranging from about 1 to 20 μg/mL. Because of the steep decrease in the curve, the resolution is poor, decreasing the accuracy in determining quantities of analyte at high concentrations and, in some cases, making it impractical or virtually impossible to determine, with any degree of accuracy, a quantity of analyte present in a sample at high concentration.

Example Lateral Flow Devices that Accurately Quantify an Analyte Present in a Sample at High Concentrations

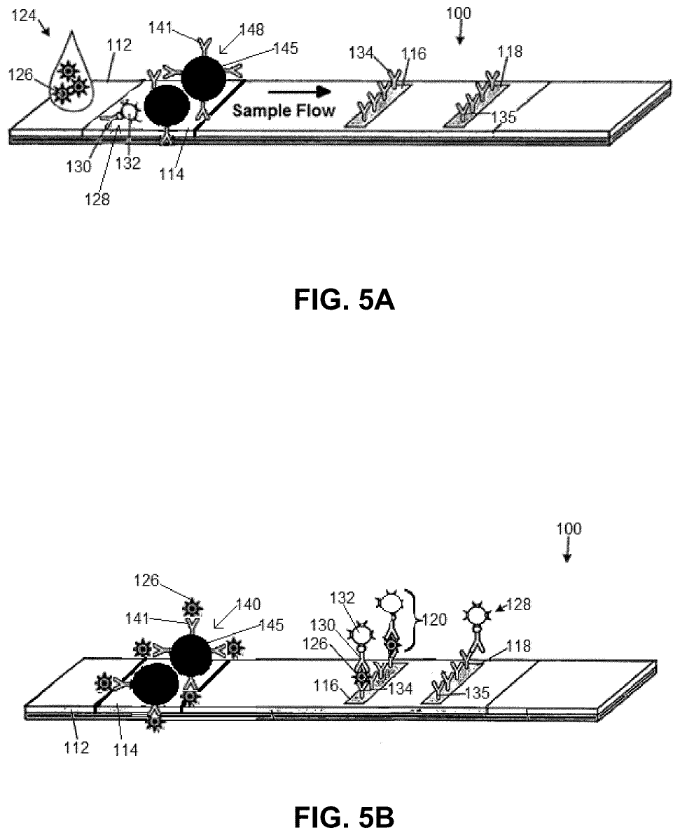

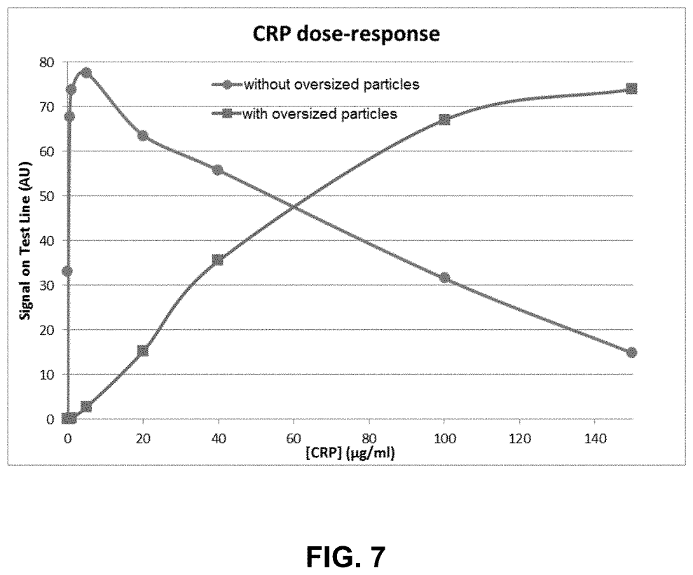

Lateral flow devices, kits, test systems, and methods described herein address these and other drawbacks of sandwich-type and competitive-type lateral flow assays, such as those illustrated in FIGS. 1A, 1B, 3A, and 3B. FIGS. 5A and 5B illustrate an example lateral flow assay 100 that can precisely measure a quantity of analyte of interest that is present in a sample, including analyte present at high concentrations. FIG. 7 is an example dose response curve that graphically illustrates the optical signal measured from the lateral flow assay 100, and specifically the relationship between a magnitude of a signal detected at the capture zone (measured along the y-axis) and the concentration of analyte in the sample applied to the assay (measured along the x-axis).

In some embodiments, such as shown in FIGS. 5A and 5B, a lateral flow assay 100 may include a test strip having a sample receiving zone 112, a label zone 114, and a capture zone 116. In some embodiments, the lateral flow assay may also include a control zone 118. FIGS. 5A and 5B illustrate the lateral flow device 100 before and after a fluid sample 124 has been applied to a sample reservoir 112. In the illustrated example, the label zone 114 is downstream of the sample receiving zone 112 along a direction of sample flow within the test strip. In some cases, the sample receiving zone 112 is located within and/or coextensive with the label zone 114.

The label zone 114 that is in or near the sample reservoir 112 includes a labeled agent 128. The labeled agent 128 can include an antibody or antibody fragment 130 bound to a label 132. A capture agent 134 is immobilized in the capture zone 116. In some cases, the antibody 130 or fragment thereof bound to the label 132 is the same type of antibody as the capture agent 134. When it is of the same type of antibody or fragment thereof, both the antibody or fragment thereof 130 bound to a label 132 and the capture agent 134 recognize and bind the same epitope of the analyte 126. In other cases, the antibody 130 or fragment thereof bound to the label 132 is a different type of antibody or fragment thereof as the capture agent 134. When it is of a different type of antibody or fragment thereof, the antibody or fragment thereof 130 bound to the label 132 recognizes and binds a different epitope of the analyte 126 from the epitope that is recognized and bound by the capture agent 134.

In some embodiments, lateral flow assays according to the present disclosure include a control line 118 downstream of the capture zone 116. A control agent 135 is immobilized in the control zone 118. The control agent 135 is specific to labeled agent that is not bound to analyte. In some cases, the control agent 135 is an antibody or fragment thereof. Labeled agent 128 that did not bind to any analyte 126 passes through and flows past the capture zone 116 (if there is excess labeled agent 128 that did not bind to analyte 126) and continues to flow down the lateral flow device 100. In lateral flow assays that include the control line 118 such as that illustrated here, the immobilized control agent 135 binds labeled agent 128 that did not bind to analyte 126 and passed through the capture zone 116 to the control line 118. In some embodiments, the control agent 135 captures the labeled agent 128 at the Fc region of the antibody. In some embodiments, the control agent 135 captures the labeled agent 128 at the Fab region of the antibody. This labeled agent 128 bound at the control line 118 emits a detectable optical signal that can be measured and used to indicate that the assay operated as intended (for example, the sample 124 flowed from the sample reservoir 112 and through the capture zone 116 as intended during normal operation of the lateral flow assay). In one implementation, the lateral flow assay 100 includes a plurality of capture zones (including at least one capture zone 116 configured to capture a labeled agent 128 according to the present disclosure), each capture zone configured to indicate the presence, absence, and/or concentration of a different analyte of interest, and a single control line 118 configured to indicate that the sample flowed through the plurality of capture zones as intended.

Embodiments of the lateral flow assay 100 according to the present disclosure also include oversize particles 145. In the implementation illustrated in FIGS. 5A and 5B, the oversize particles 145 are located in the label zone 114 but other locations upstream of the capture zone 116 may be suitable. For example, the oversize particles 145 may be located in the sample receiving zone 112 upstream of the label zone 114. According to the present disclosure, the oversize particles 145 are conjugated to a binding agent 141 specific to the analyte 126. The binding agent 141 can include, for example, an analyte-specific antibody 141 or fragment thereof. Oversize particles 145 conjugated to analyte-specific antibody 141 form antibody-conjugated oversized particles 148. As will be described in detail below, the antibody-conjugated oversized particles 148 are significantly larger in size than the labeled agent 128, and thus the features illustrated in FIGS. 5A and 5B are not drawn to scale. Additionally, although the example of FIGS. 5A and 5B is explained in the context of antibody-conjugated oversize particles 148, it will be understood that oversized particles according to the present disclosure can be conjugated to any suitable binding agent that is specific to the analyte of interest (such as but not limited to an analyte-specific antibody or fragment thereof).

The oversized particle 145 can be made of any suitable material and be of any suitable size/dimension to substantially maintain its position and resist flowing to the capture zone 116 upon application of a sample to the lateral flow device 100. In some embodiments, the oversized particle 145 is a silicon particle, a latex particle, a magnetic particle, a gold particle, or another particle of sufficient size that resists mobilizing and flowing through the assay test strip to the capture zone 116. The oversized particle 145 can include, for example, a latex bead, a magnetic bead, a silicon bead, a gold bead, or a bead formed of another suitable material. In one non-limiting example, the oversized particle 145 is around 10 μm in diameter, while the labeled agent 128 is around 40 nm in diameter. Accordingly, implementations of oversized particles described herein can have a diameter 250 times that of a conventional labeled agent. Other sizes are suitable. For example, the oversized particle 145 may be 1 to 15 μm in diameter, such as 1, 1.5, 2, 2.5, 3, 3.5, 4, 4.5, 5, 6, 7, 8, 9, 10, 11, 12, 13, 14, or 15 μm in diameter, or a diameter within a range defined by any two of the aforementioned values. The oversized particle 148 is of sufficient size that it cannot mobilize through the lateral flow device 100 but instead remains fixed at the label zone 114. In some cases the oversized particle 145 does not move upon application of a sample to the lateral flow device 100. In some cases, the oversized particle 145 may move small amounts relative to its initial position (for example, in the current non-limiting example, the oversized particle 145 may shift or change location slightly within the label zone 114). The oversized particle 145, however, does not flow to the capture zone 116 with the fluid front, as does the labeled agent 128, upon application of a sample to the lateral flow device 100. The oversized particle 145 can be integrated on the lateral flow assay 100 in a number of ways described in detail below.

In the embodiment illustrated in FIGS. 5A and 5B, the binding agent 141 conjugated to the oversize particle 145 is an antibody or fragment thereof that is specific to the analyte 126. In some cases, the antibody 141 or fragment thereof can be the same antibody type as the antibody 130 bound to the label 132 and/or the capture agent 134. In other cases, the antibody 141 or fragment thereof can be a different antibody type as the antibody 130 bound to the label 132 and/or the capture agent 134. Thus, in some embodiments, the antibody 141, antibody 130, and capture agent 134 are of the same antibody type against the analyte 126, and recognize and bind the same epitope on the analyte 126. In other embodiments, the antibody 141, antibody 130, and capture agent 134 are of a different antibody type against the analyte 126, and recognize and bind different epitopes on the analyte.

According to the present disclosure, the binding agent 141 is conjugated to the oversized particle 145 at a particular known ratio. The conjugation ratio can be adjusted depending on the particular oversized particle 145, the particular analyte of interest, the test parameters of the lateral flow device, or other parameters. In some embodiments, the binding agent 141 is present on the oversized particle 145 in a ratio of 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, or 100:1, or an amount within a range defined by any two of the aforementioned values. In one non-limiting example, the ratio of binding agent 141 to oversized particle 145 represents the binding capacity of the antibody-conjugated oversized particles 148. For example, a ratio of 1:1 means that the antibody-conjugated oversized particle 148 has a capacity of binding to a single analyte of interest in the sample, and a ratio of 100:1 means that the antibody-conjugated oversized particles 148 has a capacity of binding up to 100 analytes of interest in the sample. In another non-limiting example, the ratio of binding agent 141 conjugated to oversized particle 145 does not directly correlate to the binding capacity of the antibody-conjugated oversized particle 148. This may occur, for example, when the binding efficiency is less than 100%, as when less than 100% of the antibody conjugated to the oversized particle 145 binds to analyte of interest in the sample. In still another non-limiting example, the ratio of binding agent 141 that is present on the oversized particle 145 is less than 1:1, as when the oversized particle 145 has a high density of functional groups relative to the conjugate antibody. In such cases, the quantity of antibody needed to form antibody-conjugated oversized particles 148 is decreased. Thus, in such instances, the ratio of binding agent 141 to oversized particles 145 may be, for example, 1:2, 1:3, 1:4, 1:5, 1:6, 1:7, 1:8, 1:9, 1:10, 1:20, 1:30, 1:40, 1:50, 1:60, 1:70, 1:80, 1:90, or 1:100, or an amount within a range defined by any two of the aforementioned values.

In some advantageous embodiments of the present disclosure, the ratio of binding agent 141 to oversized particle is finely tuned and quantified in order to determine the binding capacity of the particular antibody-conjugated oversized particles 148 used for a test event on the lateral flow device 100. In addition, the amount of antibody-conjugated oversized particles 148 initially integrated onto the surface of the lateral flow device 100 is finely tuned and quantified, such that the total binding capacity of the antibody-conjugated oversized particles 148 integrated onto the surface of the lateral flow device 100 is predetermined. In non-limiting examples of the present disclosure, antibody-conjugated oversized particles 148 are integrated onto the surface of the lateral flow device 100 in an amount to bind analyte of interest in an amount of 0.05, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 40, or 50 μg/mL of analyte, or an amount within a range defined by any two of the aforementioned values. Accordingly, the antibody-conjugated oversized particles 148 can be integrated on the label zone 114 in a known quantity and available to capture a known, maximum quantity of analyte when present in the sample applied to the sample reservoir 112.

When the fluid sample 124 is applied to the sample reservoir 112, the sample 124 solubilizes the labeled agent 128 and the antibody-conjugated oversized particles 148. The labeled agent 128 binds to analyte 126, forming a label-antibody-analyte complex 120. The labeled agent 128 competes with the antibody-conjugated oversized particles 148, which also binds to analyte 126, forming an analyte-antibody-oversized particle complex 140. The label-antibody-analyte complex 120 flows through the lateral flow device 100 to the capture zone 116, where it is bound by capture agent 134, and emits a detectable signal. The analyte-antibody-oversized particle complex 140, however, does not flow through the lateral flow device 100. The complex 140 instead remains in the label zone 114, thereby retaining a quantity of bound analyte 126 and preventing the bound analyte 126 from flowing through the lateral flow device to the capture zone 116. In some implementations in which the antibody-conjugated oversized particles 148 have a known binding capacity for analyte 126, a known quantity of analyte 126 is retained in the label zone 114 when the fluid sample 124 is applied to the lateral flow device 100. For example, in some embodiments, the antibody-conjugated oversized particles 148 according to the present disclosure are capable of retaining an amount of analyte 126 in the label zone 114 of 0.05, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 40, or 50 μg/mL of analyte, or an amount within a range defined by any two of the aforementioned values.

Multiplex assays that test for the presence, absence, and/or quantity of a plurality of different analytes of interest can include a lateral flow assay according to the present disclosure (as described above with reference to FIGS. 5A and 5B) on the same test strip as one or more sandwich-type lateral flow assays as described above with reference to FIGS. 1A and 1B. In such multiplex assays, a control line may be advantageously included on the test strip to confirm that the sample has flowed through the control zone 116.

Advantages of the lateral flow device 100 will now be described with reference to FIG. 7. By retaining a known quantity of analyte 126 within the label zone 114, the lateral flow assay 100 is capable of detecting elevated concentrations of analyte 126 within the sample 124 in a single phase format. The dose response curves of FIG. 7 graphically illustrate the relationship between an intensity of a signal detected at the capture zone (measured along the y-axis in arbitrary signal intensity units) and the concentration of analyte in the sample (in this non-limiting example, C-reactive protein (CRP) measured along the x-axis in μg/ml). As illustrated in FIG. 7, the dose response curve of a lateral flow assay including antibody-conjugated oversized particles 148 according to the present disclosure (square data points) is compared to a dose response curve of a typical sandwich-type assay that does not include antibody-conjugated oversized particles 148 (circle data points).

As shown in the example of FIG. 7, the dose response curve of the sandwich-type assay shows a very steep increase in signal intensity with small increases of concentration in the range of 0.1 to 5 μg/mL of C-reactive protein (CRP), and reaches a maximum signal intensity at about 5 μg/mL of CRP. The dose response curve then decreases as free, unbound analyte competes with label-antibody-analyte complex at the capture zone, resulting in the hook effect as described above with reference to FIG. 2. In contrast, the dose response curve of the assay including antibody-conjugated oversized particles according to the present disclosure shows a gradual, single-phase increase in the optical signal strength as CRP concentration increases, allowing for a precise determination of CRP concentration at a wide range of concentrations, and significantly, at elevated concentrations, such as at concentrations greater than 5 μg/mL (the concentration at which the typical sandwich-type assay reached a maximum signal intensity).

In the example illustrated in FIG. 7, the binding capacity of the antibody-conjugated oversized particles is about 1 μg/mL. The dose response curve of the assay including antibody-conjugated oversized particles emits a signal at or near zero for concentrations less than 1 μg/mL of CRP (the binding capacity of the antibody-conjugated oversized particles in this example), after which the signal gradually increases, allowing for precise determination of analyte concentrations when analyte is present in amounts greater than 5 μg/mL.

The amount of binding agent and the binding capacity of the binding agent can be precisely tuned for the specific assay. For example, in a sample that is known or suspected of having concentrations of analyte greater than a given quantity, an amount of antibody-conjugated oversized particles of known binding capacity can be integrated on the lateral flow device such that the dose response curve resembles that shown in FIG. 7 and the concentration of analyte can be accurately determined. This is particularly advantageous in situations where the sample is suspected of having high or very high concentrations of analyte. Alternatively, or in addition, the binding capacity of the antibody-conjugated oversized particles can be precisely tuned to capture a given amount of analyte, allowing the operator to very accurately quantify the amount of analyte in the sample applied to the lateral flow device.

The antibody-conjugated oversized particle 148 can be applied to the test strip in many different ways. In the embodiment of the present disclosure illustrated in FIGS. 5A and 5B, the antibody-conjugated oversized particles 148 are pre-formed and integrated on the test strip before any fluid sample 124 has been applied to the lateral flow device 100. In one non-limiting example, the antibody-conjugated oversized particle 148 is formed and integrated onto the conjugate pad before fluid sample 124 is applied. In one non-limiting aspect, the antibody-conjugated oversized particle 148 is applied to the label zone 114 by spraying a solution of the antibody-conjugated oversized particle 148 with airjet techniques. In another non-limiting aspect, a solution including the antibody-conjugated oversized particle 148 is deposited by pouring the solution, spraying the solution, formulating the solution as a powder or gel that is placed or rubbed on the test strip, or any other suitable method to apply the antibody-conjugated oversized particle 148.

In some embodiments, after deposition, the antibody-conjugated oversized particle 148 is dried on the surface of the test strip after deposition by heating or blowing air on the conjugate pad. Other mechanisms to dry the antibody-conjugated oversized particle 148 on the surface of the test strip are suitable. For example, vacuum or lyophilization can also be used to dry the antibody-conjugated oversized particle 148 on the conjugate pad. In some cases, the antibody-conjugated oversized particle 148 is not added to a solution prior to deposition and is instead applied directly to the test strip. The antibody-conjugated oversized particle 148 can be directly applied using any suitable method, including but not limited to applying compressive or vacuum pressure to the antibody-conjugated oversized particle 148 on the surface of the test strip and/or applying the antibody-conjugated oversized particle 148 in the form of lyophilized particles to the surface of the test strip. The labeled agent 128 can be similarly deposited onto the test strip using the same or similar techniques as described for depositing the antibody-conjugated oversized particle 148.

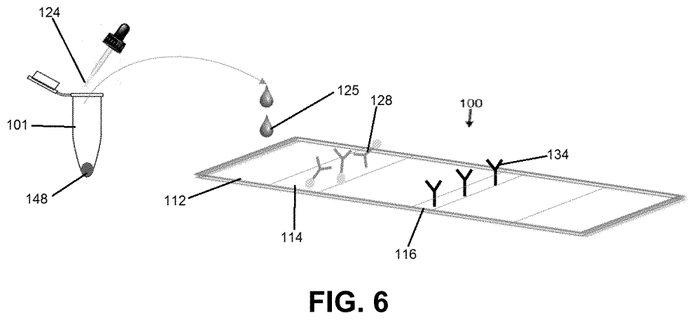

Another non-limiting example of the present disclosure will now be described with reference to FIG. 6. In contrast to the embodiment illustrated in FIGS. 5A and 5B, antibody-conjugated oversized particles 148 are not integrated onto the surface of the lateral flow device 100 before application of a sample having or suspecting of having analyte of interest 126. Instead, antibody-conjugated oversized particles 148 are applied to the lateral flow device 100 at the same time as the sample. As shown in FIG. 6, a fluid sample 124 that is to be tested for the presence, absence, or quantity of analyte of interest is added to the antibody-conjugated oversized particles 148 in a separate container 101. The fluid sample is mixed to integrate the antibody-conjugated oversized particles 148 within the fluid sample, and to allow the antibody-conjugated oversized particles 148 to bind with analyte 126, if present. Analyte 126, if present in the fluid sample 124, binds to the antibody-conjugated oversized particles 148 to form analyte-antibody-oversized particle complex 140.

A fluid sample 125 containing the complex 140 is then applied to the lateral flow device 100 at the sample reservoir 112. The lateral flow device 100 can be a conventional sandwich-type lateral flow assay device (such as that described above with reference to FIGS. 1A and 1B). The fluid sample travels to the label zone 114 where it solubilizes the labeled agent 128. The antibody-conjugated oversized particles 148 remain in the sample reservoir 112 as the fluid front moves to the label zone 114. The excessive size of the antibody-conjugated oversized particles 148 causes the antibody-conjugated oversized particles 148 to resist mobilizing from the initial location in the sample reservoir 112 where they were initially deposited when the sample 125 was applied in the sample reservoir 112. Meanwhile, in the label zone 114, the labeled agent 128 binds to any unbound analyte 126 that did not bind to the antibody-conjugated oversized particles 148 when they were mixed with the sample 124 prior to application to the device 100. The labeled agent 128 then mobilizes and travels with the fluid front to the capture zone 116, where it is captured by a capture agent 134 and generates a detectable signal.

In some cases, the detectable signal emitted at the capture zone is compared to a dose response curve specific to the test device and known quantity of antibody-conjugated oversized particles in the container 101 to determine a quantity of analyte of interest. In other cases, the quantity of analyte of interest is calculated directly by adjusting a dose response curve of the lateral flow sandwich-type assay 100 by the amount of analyte retained by antibody-conjugated oversized particles (which can be determined using the amount and conjugation ratio of the antibody-conjugated oversized particles used during the test).

Embodiments of the present disclosure that contact a sample with antibody-conjugated oversized particles include many advantages. The operator can use any conventional sandwich-type lateral flow assay test strip in embodiments of the present disclosure described with reference to FIG. 6, and still detect a precise, highly accurate quantity of analyte of interest, even when the analyte of interest is present in the sample at high or very high concentration. Embodiments of the present disclosure described with reference to FIG. 6 allow the operator the flexibility to apply antibody-conjugated oversized particles in a quantity and a conjugation ratio that is selected for the particular test the operator desires to run on the lateral flow device. Accordingly, the operator can adjust the amount of antibody-conjugated oversized particles and/or the conjugation ratio of antibody-conjugated oversized particles used during the test once the parameters of the test event (such as features of the sample to be tested) are known. Embodiments of the present disclosure described with reference to FIG. 6 may beneficially be packaged in kit form, with the lateral flow test device and antibody-conjugated oversized particles packaged in separate containers. This can result in a kit having a longer shelf life than that of embodiments of the present disclosure having antibody-conjugated oversized particles pre-integrated on the test strip.

### Example Lateral Flow Kits for Quantifying an Analyte Present in a Sample at High Concentrations

Embodiments of the present disclosure include a kit including a lateral flow device and antibody-conjugated oversized particles. Kits can also include a reader device configured to read signals from the lateral flow device and output a test result. In one non-limiting example, the kit includes a lateral flow device as described herein with reference to FIGS. 5A and 5B. The lateral flow device includes a labeled agent specific to an analyte of interest in the label zone, a capture zone including capture agent specific to the analyte of interest, and antibody-conjugated oversized particles integrated on the lateral flow device upstream of the capture zone (such as but not limited to with the labeled agent in the label zone). In some embodiments, the lateral flow device may further include a control zone, including a control agent specific to labeled agent that does not bind to analyte of interest. The kit can include instructions for use, including instructions to the operator to apply the sample to be tested to the sample reservoir of the lateral flow device. The instructions for use can instruct the operator to insert the lateral flow device into a reader (during the development time or after the development time is complete) to detect optical signals generated on the lateral flow device.

In another non-limiting example, the kit includes a lateral flow device and separately-packaged antibody-conjugated oversized particles. The lateral flow device in this example can be a conventional sandwich-type lateral flow device. The lateral flow device in this example includes a labeled agent specific to an analyte of interest in the label zone, a capture zone including capture agent specific to the analyte of interest, but does not include antibody-conjugated oversized particles pre-integrated onto the lateral flow device. Instead, antibody-conjugated oversized particles are packaged separate from the lateral flow device in the kit. In one non-limiting example, oversized particles are provided in lyophilized format in a separate package. The kit can include instructions for use, including instructions to the operator to open the package of antibody-conjugated oversized particles, add the antibody-conjugated oversized particles to a sample to be tested, optionally mix the sample, optionally allow the sample to incubate such that the antibody-conjugated oversized particles bind to analyte present in the sample, and then apply the sample containing antibody-conjugated oversized particles to the lateral flow device. The instructions for use can instruct the operator to insert the lateral flow device into a reader (during the development time or after the development time is complete) to detect optical signals generated on the lateral flow device.

### Example Lateral Flow Assay Methods to Accurately Quantify an Analyte Present in a Sample at High Concentrations

Embodiments of the present disclosure include methods of quantifying analyte present in a sample at high concentrations. In one example method, the method begins by providing a lateral flow device according to the present disclosure. The lateral flow device includes a labeled agent and antibody-conjugated oversized particles integrated on the lateral flow device prior to application of a sample. Next, a sample to be tested is applied to the lateral flow assay at the sample reservoir. Following application of the sample to the sample reservoir, the antibody-conjugated oversized particles bind analyte present in the sample (if any), competing with the labeled agent to bind to the analyte. Analyte in the sample that is not bound by the antibody-conjugated oversized particles binds to the labeled agent forming a label-agent-analyte complex. Next, the label-agent-analyte complex flows to the capture zone and is bound by capture agent, while analyte bound by antibody-conjugated oversized particles remains in the label zone (or other location on the test strip where they were initially integrated). The method continues to the next step, wherein the label-agent-analyte complex emits a detectable signal at the capture zone. Next, the detectable signal is read, by human observation or a reader device. The detected signal can be correlated to a presence, absence, or a concentration of analyte in the sample. In one example, the concentration of analyte is determined empirically, by comparing the intensity of the detectable signal to a dose response curve specific to the lateral flow device and the known quantity and binding capacity of the antibody-conjugated oversized particles that were pre-integrated on the lateral flow device.

In another example method, the method begins by providing a conventional lateral flow device (such as that described above with reference to FIGS. 1A and 1B) and a separately-packaged, known quantity of antibody-conjugated oversized particles of known binding capacity. The method begins by adding the antibody-conjugated oversized particles to the sample to be tested (or, alternatively, by adding the sample to be tested to the antibody-conjugated oversized particles). Optionally, the method can include mixing the sample and/or allowing the mixture to incubate, during which a known quantity of analyte binds to antibody-conjugated oversized particles in the mixture. The method continues to the next step, wherein the sample, including the antibody-conjugated oversized particles now bound to analyte, is applied to the lateral flow device at the sample reservoir. Next, analyte that was not bound by the antibody-conjugated oversized particles binds to labeled agent in the label zone, forming a label-agent-analyte complex. The method moves to the next step, in which label-agent-analyte complex flows to the capture zone, and is captured by capture agents, while analyte bound by antibody-conjugated oversized particles remains in the sample reservoir, and does not flow through the lateral flow device. The method continues to the next step, wherein the label-agent-analyte complex emits a detectable signal at the capture zone. Next, the detectable signal is read, by human observation or a reader device. The detected signal can be correlated to a presence, absence, or a concentration of analyte in the sample. In one example, the concentration of analyte is determined empirically, by comparing the intensity of the detectable signal to a dose response curve specific to the lateral flow device and the known quantity and binding capacity of the separately-packaged antibody-conjugated oversized particles that were initially mixed with the sample.

In some embodiments, the lateral flow device may be used to determine multiple analytes of interest in a single sample or in multiple samples in a multiplex assay format. Some embodiments of a multiplex assay include detection of a specific sub-species of a target analyte. For example, a sample may contain one or more sub-species of a target analyte. The sample may first be treated with an agent to remove one or more particular sub-species of a target analyte, and thereby lower the target analyte concentration. By way of example, a sample may contain three sub-species of a target analyte. The sample may be premixed with antibody-conjugated oversized particles that includes an antibody to remove a first sub-species of target analyte. The antibody-conjugated oversized particles bound to the first sub-species of target analyte are removed from the sample. The sample, now containing the remaining two sub-species of target analyte, is applied to a lateral flow device. A generic antibody that binds to all sub-species of the target analyte is then used as the labeled agent to recognize and bind the remaining two sub-species of the target analyte.

The following non-limiting examples illustrate features of lateral flow devices, test systems, and methods described herein, and are in no way intended to limit the scope of the present disclosure.

### Example 1

### Preparation of a Lateral Flow Assay to Quantify Elevated Protein Concentration

The following example describes preparation of a lateral flow assay to quantify an analyte of interest as described herein. In this non-limiting example, the analyte of interest is a protein, C-reactive protein (CRP), present in a serum sample at an elevated or high concentration.

CRP is a protein found in blood plasma. Levels of CRP rise in response to inflammation. CRP is thus a marker for inflammation that can be used to screen for inflammation. Elevated levels of CRP in the serum of a subject can be correlated to inflammation, viral infection, and/or bacterial infection in the subject. Normal levels of CRP in healthy human subjects range from about 1 μg/mL to about 10 μg/mL. Concentrations of CRP during mild inflammation and viral infection range from 10-40 μg/mL; during active inflammation and bacterial infection from 40-200 μg/mL; and in severe bacterial infections and burn cases greater than 200 μg/mL. Measuring and charting CRP levels be useful in determining disease progress or the effectiveness of treatments.

The assay prepared according to this non-limiting example can be used to determine the precise concentration of CRP (the analyte of interest) in a serum sample even when the concentration is above normal levels of CRP in healthy human subjects (about 1 μg/mL to about 10 μg/mL). The assay includes a labeled agent and antibody-conjugated oversized particles, and avoids several drawbacks of sandwich-type lateral flow assays, including drawbacks associated with the hook effect.

To prepare the assay, anti-C-reactive protein (anti-CRP) antibody was incubated with gold nanoparticles to form labeled anti-CRP antibody. The labeled antibody was deposited in an amount of 1.8 μL/test strip onto a conjugate pad (label zone) by spraying a solution including the labeled antibody with airjet. The conjugate pad was heated to dry the complex to the conjugate pad.

Antibody-conjugated oversized particles were prepared by first conjugating anti-CRP antibody to oversized particles. Magnetic particles (10 μm) with active surface N-hydroxysuccinimide (NHS) groups for conjugation were washed with equilibration buffer and removed from the buffer by applying a magnetic field. 200 μg of anti-CRP was placed in 60 μL PBS buffer. The antibody solution was added to the settled particles pellet and mixed for 1 hour at room temperature. The oversized particles conjugated to antibody were removed from the buffer by application of a magnetic field. The antibody-conjugated oversized particles were washed with quench buffer and incubated for 1 hour at room temperature. The quench buffer was removed, and the particles re-suspended in 100 μL PBS and stored at 4° C.

The antibody-conjugated oversized particles may be stored for later use, for example for mixing with a sample to be tested just prior to applying the sample to the lateral flow assay, or for placement directly onto the lateral flow assay prior to application of a sample. The antibody-conjugated oversized particles can be directly applied to the lateral flow assay during manufacture of the lateral flow assay, just prior to applying the sample to the lateral flow assay, or any other suitable time. In instances where the antibody-conjugated oversized particles were applied to the lateral flow assay prior to application of a sample, the solution containing the antibody-conjugated oversized particles was deposited onto the conjugate pad (label zone) by spraying the solution with airjet. The conjugate pad was heated to dry the complex to the conjugate pad.

In this example, anti-CRP antibody was deposited at the capture zone in an amount of 2 mg/mL. Goat anti-mouse antibody was deposited at a control zone in an amount of 2 mg/mL.

### Example 2

### Quantification of High Concentration C-Reactive Protein Using a Lateral Flow Assay

Due to the hook effect, sandwich-type lateral flow assays such as those described above with reference to FIGS. 1A and 1B are generally unsuitable to quantify the concentration of CRP when it is present at elevated levels in a sample. To determine elevated concentrations previously required serial dilutions of the sample, resulting in an inefficient and laborious process subject to operator error. Using lateral flow devices, kits, test systems, and methods described herein, however, concentrations of CRP above healthy levels can be accurately, reliably, and quickly quantified.

The lateral flow assay described in Example 1 that does not include antibody-conjugated oversized particles pre-integrated on the test strip was used for the following example. Antibody-conjugated oversized particles, having antibody conjugated thereon, in a storage buffer were prepared. CRP samples were prepared at different concentrations, including 0, 0.1, 0.5, 1, 5, 20, 40, 100, 150 μg/mL. Next, 20 μL of the antibody-conjugated oversized particles was placed into a tube, and the storage buffer removed by applying a magnetic field. 40 μL of CRP solution was added to the tube containing antibody-conjugated oversized particle pellet (a separate tube for each CRP sample), and the solution was mixed by agitation for about 30 seconds. Sample from each tube was applied to the sample reservoir of a conventional sandwich-type lateral flow device, and the test result was read at about two minutes.

Samples that were not treated with antibody-conjugated oversized particles were similarly prepared in the concentrations described above. 40 μL of each sample was applied to the sample reservoir of a conventional sandwich-type lateral flow device, and the test result was read at about ten minutes.

In accordance with advantageous features of the present disclosure, the amount of antibody-conjugated oversized particles used to bind to CRP was carefully considered to ensure binding to a requisite amount of CRP to provide an optimal range of signals at the capture zone that allowed a single rising phase dose response in the test system, enabling quantification of elevated levels of CRP. Insufficient amounts of antibody-conjugated oversized particles (or insufficient anti-CRP conjugated to the oversized particles) can result in a steep rising phase of the dose response curve, which can results in insufficient resolution for determination of the CRP concentration. Excessive antibody-conjugated oversized particles (or excessive anti-CRP conjugated to the oversized particle) results in no or low signal intensity until the antibody-conjugated oversized particle is saturated, at which point signal intensity increases, making it difficult to determine the concentration of analyte at the lower range of analyte concentration. Accordingly, the amount of antibody-conjugated oversized particles added to the sample or pre-deposited on the lateral flow device can be advantageously varied to accommodate the requirement of different concentration ranges of analytes.

A lateral flow assay including antibody-conjugated oversized particles deposited in a suitable location upstream of the capture zone prior to sample application can also be used similarly. In such cases, the sample does not require mixture with or pre-incubation with the antibody-conjugated oversized particles, but instead is applied directly to the lateral flow assay that includes antibody-conjugated oversized particles pre-integrated on a surface of the lateral flow assay.

The results of Example 2 are presented in FIG. 7, which illustrates the resulting dose response curves for the lateral flow assays with antibody-conjugated oversized particles (line with square data points) and the lateral flow assays without antibody-conjugated oversized particles (conventional sandwich-type lateral flow assay; line with circle data points). The test results are also described in Table 1.

TABLE 1
Comparison of Traditional Sandwich-Type Lateral Flow Assay
and Lateral Flow Assay of the Present Disclosure

| | Signal (AU) of sample | Signal (AU) of sample |
|---|---|---|
| Amount of Unlabeled | with antibody- | without antibody- |
| CRP (μg/mL) in | conjugated oversized | conjugated oversized |
| Serum Sample | particle | particle |
| 0 | 0 | 0 |
| 0.1 | 0 | 33.14 |
| 0.5 | 0 | 67.63 |
| 1 | 0.19 | 73.72 |
| 5 | 2.75 | 77.53 |
| 20 | 15.21 | 63.63 |
| 40 | 35.50 | 55.78 |
| 100 | 67.07 | 31.55 |
| 150 | 73.95 | 14.82 |

As shown in Table 1, the signal intensity for the sample without antibody-conjugated oversized particles has a steep increase from a concentration of 0 to 5 μg/mL. The signal decreases with increasing concentration in amounts greater than 5 μg/mL. In addition, the signal observed at 0.1 μg/mL (33.14 AU) is similar to the signal observed at 100 μg/mL (31.55). Thus, if a sample is obtained that results in a signal intensity of around 31-33 AU, it is unclear whether that signal corresponds to CRP in a healthy amount (around 0.1 μg/mL) or in an unhealthy amount (100 μg/mL).

In contrast, the lateral flow assay according to the present disclosure allows the concentration of CRP to be accurately determined at concentrations greater than 5 μg/mL. This is particularly advantageous in the present example where the analyte of interest is CRP, which elevates to concentrations greater than 10 μg/mL when inflammation or disease conditions are present. In contrast to the sandwich-type lateral flow assay that does not include antibody-conjugated oversized particles, where it is not possible to determine the concentration of CRP greater than 5 μg/mL, the lateral flow assay with antibody-conjugated oversized particles allows precise determination of the concentration by providing a single rising phase dose response curve with sufficient resolution to accurately quantify the CRP concentration. Embodiments of the lateral flow assays described herein allow a user to determine with confidence that the concentration of CRP in the subject being tested is above normal levels. When a test according to the present disclosure is performed and indicates a concentration of CRP greater than healthy levels (for example, greater than 10 μg/mL), this information can be correlated to an inflammation, a viral infection, and/or a bacterial infection condition.

Further, the ability to accurately pinpoint the precise concentration of CRP in the subject under test can allow the test result to be correlated to a specific type of disease condition. For example, a concentration between 10 μg/mL and 20 μg/mL may be correlated to mild inflammation whereas a concentration between 40 μg/mL and 200 μg/mL may be correlated to a bacterial infection. In addition, the ability to accurately pinpoint the precise concentration of CRP in the subject under test may allow the test result to be correlated to a stage of disease. For example, a concentration between 40 μg/mL and 200 μg/mL may be correlated to mild bacterial infection whereas a concentration greater than 200 μg/mL may be correlated to a severe bacterial infection. These examples are illustrative and are not intended to limit the scope of the present disclosure.

Furthermore, lateral flow devices described herein quantify elevated concentrations of an analyte in a sample in one single assay, without the need to dilute the sample. Assays such as those described with reference to FIGS. 1A, 1B, 3A, and 3B, in contrast, require dilution of samples that include high concentrations of analyte; otherwise, the signals of the high-concentration portion of the dose response curve are indistinguishable. The lateral flow assay of the present disclosure is capable of determining even minute differences in elevated analyte concentration based on a single signal obtained at the capture zone after one test.

Embodiments of the lateral flow devices herein (including oversized particles) can be advantageously combined with conventional sandwich-type lateral flow devices (that do not include oversized particles) to extend the range of concentrations that can be accurately and precisely tested. As shown in FIG. 7, in some embodiments of lateral flow devices described herein, resolution of signals at very low concentrations of analyte may be low. In cases where the concentration of the analyte of interest is very low, the lateral flow device including oversized particles may still indicate the presence or absence of the analyte of interest but may not provide an optimal quantitative measurement of the very low concentration analyte. In cases where the unknown concentration of the analyte of interest may fall within a large range of concentrations, the sample can be divided into two portions. The first sample portion can be mixed with oversized particles and applied to a first lateral flow device (or applied to a first lateral flow device with integrated oversized particles as described herein). The second sample portion can be applied to a second conventional lateral flow device (without oversized particles). The results of the test of the first lateral flow device in combination with the results of the test of the second lateral flow device can give the operator a very accurate measurement of the actual concentration of the analyte of interest over a very broad range of possible concentrations.

### Methods of Diagnosing a Condition Using Lateral Flow Assays According to the Present Disclosure

Some embodiments provided herein relate to methods of using lateral flow assays to diagnose a medical condition. In some embodiments, the method includes providing a lateral flow assay and antibody-conjugated oversized particles (packaged separately or pre-integrated on the lateral flow assay) as described herein. In some embodiments, the method includes receiving a sample at a sample reservoir of the lateral flow assay.

In some embodiments, the sample is obtained from a source, including an environmental or biological source. In some embodiments, the sample is suspected of having an analyte of interest. In some embodiments, the sample is not suspected of having an analyte of interest. In some embodiments, a sample is obtained and analyzed for verification of the absence or presence of an analyte. In some embodiments, a sample is obtained and analyzed for the quantity of analyte in the sample. In some embodiments, the quantity of an analyte in a sample is less than a normal value present in healthy subjects, at or around a normal value present in healthy subjects, or above a normal value present in healthy subjects.

In some embodiments, receiving a sample at the sample reservoir of the lateral flow assay includes contacting a sample with a lateral flow assay. A sample may contact a lateral flow assay by introducing a sample to a sample reservoir by external application, as with a dropper or other applicator. In some embodiments, a sample reservoir may be directly immersed in the sample, such as when a test strip is dipped into a container holding a sample. In some embodiments, a sample may be poured, dripped, sprayed, placed, or otherwise contacted with the sample reservoir.

A labeled agent in embodiments of the present disclosure include an antibody, and a label and can be deposited on a conjugate pad (or label zone) within or downstream of the sample reservoir. The labeled agent can be integrated on the conjugate pad by physical or chemical bonds. The binding particle may also be integrated on the conjugate pad by physical or chemical bonds, or alternatively may be added to the sample prior to application of the sample on the lateral flow device. Antibody-conjugated oversized particles recognize and bind to analyte in the sample, forming analyte-antibody-oversized particle complex (whether the antibody-conjugated oversized particles are first added to the sample or whether the sample is added to the lateral flow assay having antibody-conjugated oversized particles integrated thereon). The sample solubilizes the labeled agent after the sample is added to the sample reservoir, releasing the bonds holding the labeled agent to the conjugate pad. Labeled agent recognizes and binds to analyte that is not bound by antibody-conjugated oversized particles, forming a label-agent-analyte complex. The sample, including the label-agent-analyte complex flow along the fluid front through the lateral flow assay to a capture zone, whereas the analyte-antibody-oversized particle complex remains in the location the antibody-conjugated oversized particle was initially deposited on the test strip (either by application of the sample onto the test strip or during integration directly onto a surface of the test strip prior to sample application).

Capture agent immobilized at the capture zone binds label-agent-analyte complex. When label-agent-analyte complex binds to capture agent at the capture zone, a detectable signal from the label is emitted. The signal may include an optical signal as described herein. When low concentrations of analyte are present in the sample (such as levels at or below healthy levels), antibody-conjugated oversized particle binds all or substantially all of the analyte in the sample, and no signal is detected at the capture zone. At elevated concentrations of analyte (such as levels above healthy values), the intensity of the detected signal increases in an amount proportionate to the amount of analyte in the sample. The detected signal and the binding capacity of the antibody-conjugated oversized particle (which includes a known quantity of bound analyte) are compared to values on a dose response curve for the analyte of interest, and the concentration of analyte in the sample is determined.

In some embodiments, the analyte is present in elevated concentrations. Elevated concentrations of analyte can refer to a concentration of analyte that is above healthy levels. Thus, elevated concentration of analyte can include a concentration of analyte that is 5%, 10%, 15%, 20%, 25%, 30%, 35%, 40%, 50%, 60%, 70%, 80%, 90%, 100%, 125%, 150%, 200%, or greater than a healthy level. In some embodiments, an analyte of interest includes C-reactive protein (CRP), which is present in blood serum of healthy individuals in an amount of about 1 to about 10 μg/mL. Thus, elevated concentrations of CRP in a sample includes an amount of 15, 20, 25, 30, 35, 40, 45, 50, 55, 60, 65, 70, 75, 80, 85, 90, 95, 100, 110, 120, 130, 140, 150, 160, 170, 180, 190, or 200 μg/mL or greater. The level at which an analyte of interest will be considered elevated may differ depending on the specific analyte of interest.

In some embodiments, upon determination that an analyte is present in a sample in elevated concentrations, the subject is diagnosed with a certain disease. In some embodiments, diagnosis of an infection is made when the concentration of CRP is determined to be 15, 20, 25, 30, 35, 40, 45, 50, 55, 60, 65, 70, 75, 80, 85, 90, 95, 100, 110, 120, 130, 140, 150, 160, 170, 180, 190, or 200 μg/mL or greater. In some embodiments, a determination that the concentration is greater than 200 μg/mL, for example, 400-500 μg/mL, results in a diagnosis of severe bacterial infection.

In some embodiments, when a sample is placed on a lateral flow device described herein, a detected signal is determined at the capture zone, and the detected signal in combination with the binding capacity of the antibody-conjugated oversized particle is used to determine the concentration of analyte in the sample. In some embodiments, when the test system determines a certain signal intensity, the test system outputs a test result. The test result can include a concentration of analyte in the sample and/or a verbal or written indication that the test result is “normal” or “within normal levels” or that the test result is “not within normal levels,” “elevated,” “very elevated” or some other indication.

### Additional Embodiments of the Present Disclosure

Embodiments of the present disclosure have been described with reference to oversized particles conjugated to binding agent. As described herein, the oversized particles are significantly larger than other components of the lateral flow device that flow through the test strip upon application of a fluid sample. For example, oversized particles described herein can be significantly larger than labeled agents that are sized and dimensioned to solubilize and mobilize from the label zone to the capture zone when a fluid sample is applied to the sample well. In contrast, the oversized particles according to the present disclosure resist motion when the fluid sample is applied, and remain in substantially the same location despite fluid flow through the test strip. Although implementations of the oversized particles are described herein with reference to their significantly large size, it will be understood that other particles that resist motion through the test strip can be implemented in accordance with the present disclosure. For example, an inertial particle can be conjugated to a binding agent and applied to a lateral flow device in the manner described herein with reference to an oversized particle. In a first non-limiting embodiment, an inertial particle is the same size as or smaller than labeled agents described herein. To illustrate with an example, a magnetic particle can be conjugated to a binding agent to form an antibody-conjugated magnetic inertial particle that is the same size as or even smaller than the labeled agent. In this example, the antibody-conjugated magnetic inertial particle can be retained in place upstream of the capture zone by application of a magnetic force to the magnetic inertial particle, thus preventing it from moving downstream to the capture zone with the fluid sample. The magnetic force can be a magnetic field applied above or below or within the test strip during sample application. In a second non-limiting embodiment, the inertial particle may be smaller in size, around the same size, or slightly larger in size than the labeled agent but be significantly denser than the labeled agent. For example, the inertial particle may be 2 times, 3 times, 4 times, 5 times, 6 times, 7 times, 8 times, 9 times, 10 times or another multiple more dense than the labeled agent. As a result in this significantly increased density, the inertial particle resists motion through the test strip from an initial location to the capture zone when a sample is applied to the test strip.

Other features in addition to or in lieu of increased density (such as but not limited to surface features that stick to or hook into nitrocellulose fibers of the conjugate pad) may be suitable to ensure the antibody-conjugated particle does not mobilize through the test strip. Features that increase the coefficient of friction of the antibody-conjugated particle may also be implemented. Accordingly, the following embodiments explaining the present disclosure in the context of an oversized particle should not be limited to an oversized particle.

In addition, other structures in addition to or in lieu of oversized particles can be implemented in accordance with the present disclosure in order to retain an amount of analyte of interest in the sample well and/or label zone so that a portion of the analyte in the sample does not flow to the capture zone. In one non-limiting example, a filter is positioned in the fluid flow path between the sample well and the capture zone (for example, at or near the label zone) to capture oversized particles bound to analyte that happen to begin mobilizing toward the capture zone. The filter can be a size exclusion filter, allowing fluid and particles of a selected size or smaller to pass through but not particles greater than the selected size. Through holes in the filter can be sized to allow labeled agent bound to analyte and the fluid sample to flow through the filer to the capture zone, but not allow oversized particles to flow through the filter, thereby retaining analye of interest upstream of the filter. Other implementations are also suitable.

### Example Test Systems Including Lateral Flow Assays According to the Present Disclosure

Lateral flow assay test systems described herein can include a lateral flow assay test device (such as but not limited to a test strip), a housing including a port configured to receive all or a portion of the test device, a reader including a light source and a light detector, a data analyzer, and combinations thereof. A housing may be made of any one of a wide variety of materials, including plastic, metal, or composite materials. The housing forms a protective enclosure for components of the diagnostic test system. The housing also defines a receptacle that mechanically registers the test strip with respect to the reader. The receptacle may be designed to receive any one of a wide variety of different types of test strips. In some embodiments, the housing is a portable device that allows for the ability to perform a lateral flow assay in a variety of environments, including on the bench, in the field, in the home, or in a facility for domestic, commercial, or environmental applications.

A reader may include one or more optoelectronic components for optically inspecting the exposed areas of the capture zone of the test strip. In some implementations, the reader includes at least one light source and at least one light detector. In some embodiments, the light source may include a semiconductor light-emitting diode and the light detector may include a semiconductor photodiode. Depending on the nature of the label that is used by the test strip, the light source may be designed to emit light within a particular wavelength range or light with a particular polarization. For example, if the label is a fluorescent label, such as a quantum dot, the light source would be designed to illuminate the exposed areas of the capture zone of the test strip with light in a wavelength range that induces fluorescent emission from the label. Similarly, the light detector may be designed to selectively capture light from the exposed areas of the capture zone. For example, if the label is a fluorescent label, the light detector would be designed to selectively capture light within the wavelength range of the fluorescent light emitted by the label or with light of a particular polarization. On the other hand, if the label is a reflective-type label, the light detector would be designed to selectively capture light within the wavelength range of the light emitted by the light source. To these ends, the light detector may include one or more optical filters that define the wavelength ranges or polarizations axes of the captured light. A signal from a label can be analyzed, using visual observation or a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of an analyte of interest can be performed using a spectrophotometer. Lateral flow assays described herein can be automated or performed robotically, if desired, and the signal from multiple samples can be detected simultaneously. Furthermore, multiple signals can be detected in a multiplex-type assay, where more than one analyte of interest is detected, identified, or quantified.

The data analyzer processes the signal measurements that are obtained by the reader. In general, the data analyzer may be implemented in any computing or processing environment, including in digital electronic circuitry or in computer hardware, firmware, or software. In some embodiments, the data analyzer includes a processor (e.g., a microcontroller, a microprocessor, or ASIC) and an analog-to-digital converter. The data analyzer can be incorporated within the housing of the diagnostic test system. In other embodiments, the data analyzer is located in a separate device, such as a computer, that may communicate with the diagnostic test system over a wired or wireless connection. The data analyzer may also include circuits for transfer of results via a wireless connection to an external source for data analysis or for reviewing the results.

In general, the results indicator may include any one of a wide variety of different mechanisms for indicating one or more results of an assay test. In some implementations, the results indicator includes one or more lights (e.g., light-emitting diodes) that are activated to indicate, for example, the completion of the assay test. In other implementations, the results indicator includes an alphanumeric display (e.g., a two or three character light-emitting diode array) for presenting assay test results.

Test systems described herein can include a power supply that supplies power to the active components of the diagnostic test system, including the reader, the data analyzer, and the results indicator. The power supply may be implemented by, for example, a replaceable battery or a rechargeable battery. In other embodiments, the diagnostic test system may be powered by an external host device (e.g., a computer connected by a USB cable).

### Features of Example Lateral Flow Devices

Lateral flow devices described herein can include a sample reservoir (also referred to as a sample receiving zone) where a fluid sample is introduced to a test strip, such as but not limited to an immunochromatographic test strip present in a lateral flow device. In one example, the sample may be introduced to sample reservoir by external application, as with a dropper or other applicator. The sample may be poured or expressed onto the sample reservoir. In another example, the sample reservoir may be directly immersed in the sample, such as when a test strip is dipped into a container holding a sample.

Lateral flow devices described herein can include a solid support or substrate. Suitable solid supports include but are not limited to nitrocellulose, the walls of wells of a reaction tray, multi-well plates, test tubes, polystyrene beads, magnetic beads, membranes, and microparticles (such as latex particles). Any suitable porous material with sufficient porosity to allow access by labeled agents and a suitable surface affinity to immobilize capture agents can be used in lateral flow devices described herein. For example, the porous structure of nitrocellulose has excellent absorption and adsorption qualities for a wide variety of reagents, for instance, capture agents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

Lateral flow devices described herein can include porous solid supports, such as nitrocellulose, in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (for example, 50 to 300 sec/4 cm), about 22.5 to 62.5 sec/cm (for example, 90 to 250 sec/4 cm), about 25 to 62.5 sec/cm (for example, 100 to 250 sec/4 cm), about 37.5 to 62.5 sec/cm (for example, 150 to 250 sec/4 cm), or about 50 to 62.5 sec/cm (for example, 200 to 250 sec/4 cm). In specific embodiments of devices described herein, the flow rate is about 35 sec/cm (for example, 140 sec/4 cm). In other specific embodiments of devices described herein, the flow rate is about 37.5 sec/cm (for example, 150 sec/4 cm).

The surface of a solid support may be activated by chemical processes that cause covalent linkage of an agent (e.g., a capture reagent) to the support. As described below, the solid support can include a conjugate pad. Many other suitable methods may be used for immobilizing an agent (e.g., a capture reagent) to a solid support including, without limitation, ionic interactions, hydrophobic interactions, covalent interactions and the like.

Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

Lateral flow devices described herein can include a conjugate pad, such as a membrane or other type of material that includes a capture reagent. The conjugate pad can be a cellulose acetate, cellulose nitrate, polyamide, polycarbonate, glass fiber, membrane, polyethersulfone, regenerated cellulose (RC), polytetra-fluorethylene, (PTFE), Polyester (e.g. Polyethylene Terephthalate), Polycarbonate (e.g., 4,4-hydroxy-diphenyl-2,2′-propane), Aluminum Oxide, Mixed Cellulose Ester (e.g., mixture of cellulose acetate and cellulose nitrate), Nylon (e.g., Polyamide, Hexamethylene-diamine, and Nylon 66), Polypropylene, PVDF, High Density Polyethylene (HDPE)+nucleating agent “aluminum dibenzoate” (DBS) (e.g. 80 u 0.024 HDPE DBS (Porex)), and HDPE. Examples of conjugate pads also include, Cyclopore® (Polyethylene terephthalate), Nucleopore® (Polyethylene terephthalate), Membra-Fil® (Cellulose Acetate and Nitrate), Whatman® (Cellulose Acetate and Nitrate), Whatman #12-S (rayon)), Anopore® (Aluminum Oxide), Anodise® (Aluminum Oxide), Sartorius (cellulose acetate, e.g. 5 μm), and Whatman Standard 17 (bound glass).

Lateral flow devices described herein are highly sensitive to an analyte of interest that is present in a sample at high concentrations. As described above, high concentrations are present when unlabeled analyte of interest in the sample is present in an amount sufficient to compete with a labeled compound to bind to a capture agent in the capture zone, resulting in a detected signal on a negative-slope portion of a dose response curve (for example, on the “hook effect” portion of the dose response curve of a conventional sandwich-type lateral flow assay or a negative-slope portion of a dose response curve according to lateral flow assays of the present disclosure). “Sensitivity” refers to the proportion of actual positives which are correctly identified as such (for example, the percentage of infected, latent or symptomatic subjects who are correctly identified as having a condition). Sensitivity may be calculated as the number of true positives divided by the sum of the number of true positives and the number of false negatives.

Lateral flow devices described herein can accurately measure an analyte of interest in many different kinds of samples. Samples can include a specimen or culture obtained from any source, as well as biological and environmental samples. Biological samples may be obtained from animals (including humans) and encompass fluids, solids, tissues, and gases. Biological samples include urine, saliva, and blood products, such as plasma, serum and the like. Such examples are not however to be construed as limiting the sample types applicable to the present disclosure.

In some embodiments the sample is an environmental sample for detecting an analyte in the environment. In some embodiments, the sample is a biological sample from a subject. In some embodiments, a biological sample can include peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, or other lavage fluids.

As used herein, “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, and metabolites of or antibodies to any of the above substances. Specific examples of some analytes include ferritin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Additional analytes may be included for purposes of biological or environmental substances of interest.

Lateral flow devices described herein can include a label. Labels can take many different forms, including a molecule or composition bound or capable of being bound to an analyte, analyte analog, detector reagent, or binding partner that is detectable by spectroscopic, photochemical, biochemical, immunochemical, electrical, optical or chemical means. Examples of labels include enzymes, colloidal metallic particles (also referred to as metallic nanoparticles, including, for example, gold, silver, copper, palladium, platinum, cadmium, or composites thereof), colored latex particles, radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulfur particles, protein-adsorbed tellurium particles, protein-adsorbed carbon particles, and protein-coupled dye sacs. The attachment of a compound (e.g., a detector reagent) to a label can be through covalent bonds, adsorption processes, hydrophobic and/or electrostatic bonds, as in chelates and the like, or combinations of these bonds and interactions and/or may involve a linking group.

The term “specific binding partner (or binding partner)” refers to a member of a pair of molecules that interacts by means of specific, noncovalent interactions that depend on the three-dimensional structures of the molecules involved. Typical pairs of specific binding partners include antigen/antibody, hapten/antibody, hormone/receptor, nucleic acid strand/complementary nucleic acid strand, substrate/enzyme, inhibitor/enzyme, carbohydrate/lectin, biotin/(strept)avidin, receptor/ligands, and virus/cellular receptor, or various combinations thereof

As used herein, the terms “immunoglobulin” or “antibody” refer to proteins that bind a specific antigen. Immunoglobulins include, but are not limited to, polyclonal, monoclonal, chimeric, and humanized antibodies, Fab fragments, F(ab′)2 fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

Lateral flow devices described herein include a labeled agent. The labeled agent can be specific for an analyte. In some embodiments, a labeled agent can be an antibody or fragment thereof that has been conjugated to, bound to, or associated with a label.

Lateral flow devices according to the present disclosure include a capture agent. A capture agent includes an immobilized agent that is capable of binding to an analyte, including a label-agent-analyte complex. A capture agent includes an unlabeled specific binding partner that is specific for (i) a label-agent-analyte complex of interest, (ii) a label-agent-analyte complex or an unbound analyte, as in a competitive assay, or for (iii) an ancillary specific binding partner, which itself is specific for the analyte, as in an indirect assay. As used herein, an “ancillary specific binding partner” is a specific binding partner that binds to the specific binding partner of an analyte. For example, an ancillary specific binding partner may include an antibody specific for another antibody, for example, goat anti-human antibody. Lateral flow devices described herein can include a “capture area” that is a region of the lateral flow device where the capture reagent is immobilized. Lateral flow devices described herein may include more than one capture area, for example, a “primary capture area,” a “secondary capture area,” and so on. In some cases, a different capture reagent will be immobilized in the primary, secondary, and/or other capture areas. Multiple capture areas may have any orientation with respect to each other on the lateral flow substrate; for example, a primary capture area may be distal or proximal to a secondary (or other) capture area along the path of fluid flow and vice versa. Alternatively, a primary capture area and a secondary (or other) capture area may be aligned along an axis perpendicular to the path of fluid flow such that fluid contacts the capture areas at the same time or about the same time.

Lateral flow devices according to the present disclosure include capture agents that are immobilized such that movement of the capture agent is restricted during normal operation of the lateral flow device. For example, movement of an immobilized capture agent is restricted before and after a fluid sample is applied to the lateral flow device. Immobilization of capture agents can be accomplished by physical means such as barriers, electrostatic interactions, hydrogen-bonding, bioaffinity, covalent interactions or combinations thereof.

Lateral flow devices according to the present disclosure can include multiplex assays. Multiplex assays include assays in which multiple, different analytes of interest can be detected, identified, and in some cases quantified. For example, in a multiplex assay device, a primary, secondary, or more capture areas may be present, each specific for one analyte of interest of a plurality of analytes of interest.

Lateral flow devices according to the present disclosure can detect, identify, and quantify a biologic. A biologic includes chemical or biochemical compounds produced by a living organism which can include a prokaryotic cell line, a eukaryotic cell line, a mammalian cell line, a microbial cell line, an insect cell line, a plant cell line, a mixed cell line, a naturally occurring cell line, or a synthetically engineered cell line. A biologic can include large macromolecules such as proteins, polysaccharides, lipids, and nucleic acids, as well as small molecules such as primary metabolites, secondary metabolites, and natural products.

It is to be understood that the description, specific examples and data, while indicating exemplary embodiments, are given by way of illustration and are not intended to limit the various embodiments of the present disclosure. Various changes and modifications within the present disclosure will become apparent to the skilled artisan from the description and data contained herein, and thus are considered part of the various embodiments of this disclosure.

What is claimed is:

1. An assay test strip comprising:
a flow path configured to receive a fluid sample;
a sample receiving zone coupled to the flow path;
a capture zone coupled to the flow path downstream of the sample receiving zone and comprising an immobilized capture agent specific to an analyte of interest;
a labeled antibody or fragment thereof coupled to the flow path upstream of the capture zone specific to the analyte of interest; and
oversized particles in the flow path upstream of the capture zone, the oversized particles conjugated to an antibody or fragment thereof specific to the analyte of interest to form antibody-conjugated oversized particles of a size and dimension to remain upstream of the capture zone when the fluid sample is received on the assay test strip.

2. The assay test strip of claim 1, wherein the flow path is configured to receive a fluid sample comprising the analyte of interest, and wherein the labeled antibody or fragment thereof and the antibody-conjugated oversized particles compete to specifically bind the analyte of interest.

3. The assay test strip of claim 2, wherein the labeled antibody or fragment thereof is configured to flow with bound analyte of interest in the flow path to the capture zone when the fluid sample is received on the assay test strip.

4. The assay test strip of claim 3, wherein the labeled antibody bound to the analyte of interest is captured at the capture zone and emits a detectable signal.

5. The assay test strip of claim 1, wherein the flow path is configured to receive a fluid sample that does or does not comprise analyte of interest, and wherein the antibody-conjugated oversized particles specifically bind to a known quantity of analyte of interest, thereby retaining a known quantity of analyte of interest upstream of the capture zone.

6. The assay test strip of claim 1, further comprising a control zone downstream of the capture zone, wherein the control zone comprises antibody that specifically binds to the labeled antibody or fragment thereof that does not bind to analyte of interest and flows past the capture zone.

7. The assay test strip of claim 1, wherein, when the fluid sample does not comprise an analyte of interest, the labeled antibody or fragment thereof flows to the control zone and emits an optical signal at the control zone only, indicating absence of the analyte of interest in the fluid sample.

8. The assay test strip of claim 1, wherein the immobilized capture agent comprises an antibody or a fragment thereof specific to the analyte of interest.

9. The assay test strip of claim 1, wherein the antibody-conjugated oversized particles are integrated onto a surface of the test strip.

10. The assay test strip of claim 1, wherein the oversized particles comprise gold particles, latex beads, magnetic beads, or silicon beads.

11. The assay test strip of claim 1, wherein the oversized particle is about 1 μm to about 15 μm in diameter.

12. The assay test strip of claim 1, wherein the fluid sample is selected from the group consisting of a blood, plasma, urine, sweat, or saliva sample.

13. The assay test strip of claim 1, wherein the analyte of interest comprises C-reactive protein (CRP) and the antibody or fragment thereof conjugated to the oversized particle comprises an anti-CRP antibody or fragment thereof bound to the CRP.

14. A diagnostic test system comprising:
a kit comprising:
an assay test strip comprising:
a flow path configured to receive a fluid sample;
a sample receiving zone coupled to the flow path;
a capture zone coupled to the flow path downstream of the sample receiving zone and comprising an immobilized capture agent specific to an analyte of interest; and
a labeled antibody or fragment thereof coupled to the flow path upstream of the capture zone specific to the analyte of interest; and
oversized particles in the flow path upstream of the capture zone, the oversized particles conjugated to an antibody or fragment thereof specific to the analyte of interest to form antibody-conjugated oversized particles of a size and dimension to remain upstream of the capture zone when the fluid sample is received on the assay test strip;
a reader comprising a light source and a detector; and
a data analyzer.

15. The system of claim 14, wherein the labeled antibody or fragment thereof is configured to flow with bound analyte of interest in the flow path to the capture zone when the fluid sample is received on the assay test strip, wherein the labeled antibody bound to the analyte of interest is captured at the capture zone and emits a detectable signal.

16. A method of determining a concentration of analyte of interest in a fluid sample comprising:
applying the fluid sample to an assay test strip comprising:
a flow path configured to receive a fluid sample;
a sample receiving zone coupled to the flow path;
a capture zone coupled to the flow path downstream of the sample receiving zone and comprising an immobilized capture agent specific to an analyte of interest;
a labeled antibody or fragment thereof coupled to the flow path upstream of the capture zone specific to the analyte of interest; and
oversized particles in the flow path upstream of the capture zone, the oversized particles conjugated to an antibody or fragment thereof specific to the analyte of interest to form antibody-conjugated oversized particles of a size and dimension to remain upstream of the capture zone when the fluid sample is received on the assay test strip;
binding analyte present in the fluid sample to labeled antibody or fragment thereof;
binding analyte present in the fluid sample to the antibody-conjugated oversized particles;
flowing the fluid sample and labeled antibody bound to analyte in the flow path to the capture zone while the antibody-conjugated oversized particles bound to analyte does not flow in the flow path to the capture zone;
binding the labeled antibody bound to analyte to the immobilized capture agent in the capture zone;
detecting a signal from the labeled antibody bound to analyte immobilized in the capture zone; and
determining the concentration of analyte based at least on the detected signal.

17. The method of claim 16, wherein the concentration is determined based on the detected signal and the quantity of antibody-conjugated oversized particles on the assay test strip.

18. The method of claim 16, wherein the detected signal is an optical signal, a fluorescence signal, or a magnetic signal.

19. The method of claim 16, further comprising displaying an indication that the analyte of interest is present in the fluid sample or a quantity of analyte of interest in the fluid sample.

20. The method of claim 16, further comprising displaying an indication that the analyte of interest is present in elevated amounts.
